(19) United States
(12) Patent Application Publication
(10) Pub. No.: US 2022/0275401 A1
(43) Pub. Date: Sep. 1, 2022

(54) METHOD FOR ENCODING DNA/RNA SEQUENCES BASED ON BIDIRECTIONAL TRINUCLEOTIDE POSITION-SPECIFIC PROPENSITIES AND POINTWISE JOINT MUTUAL INFORMATION

(71) Applicant: Shaanxi Normal University, Xi'an City (CN)

(72) Inventors: Juanying XIE, Xi'an City (CN); Mingzhao WANG, Xi'an City (CN); Shengquan XU, Xi'an City (CN)

(21) Appl. No.: 17/522,237

(22) Filed: Nov. 9, 2021

(30) Foreign Application Priority Data
Nov. 9, 2020 (CN) 202011236108.2

(51) Int. Cl. C12N 15/90; C12N 9/22; C12N 15/113; C12N 15/63

(52) U.S. Cl. CPC C12N 15/907; C12N 15/63; C12N 15/113; C12N 9/22

(57) ABSTRACT

Disclosed is a method for encoding DNA/RNA sequences based on bidirectional trinucleotide position-specific propensities and pointwise joint mutual information, which consists of the steps: constructing the nucleotide position-specific propensity matrix of DNA/RNA sequences; constructing the bidirectional dinucleotide position-specific propensity matrix of DNA/RNA sequences; constructing the bidirectional trinucleotide position-specific propensity matrix of DNA/RNA sequences; determining the value of pointwise joint mutual information of the nucleotides of DNA/RNA sequences; concatenating features and encoding DNA/RNA sequences. In order to extract more position information of trinucleotides from DNA/RNA sequences, a parameter β is introduced to represent the distance between the current nucleotide and its forward or backward adjacent dinucleotide, the numerical feature vectors obtained from different values of β are concatenated into a high-dimensional numerical feature vector.

This patent application claims the benefit and priority of Chinese Patent Application No. 202011236108.2 entitled “Method for encoding DNA/RNA sequences based on bidirectional trinucleotide position-specific propensities and pointwise joint mutual information” filed on Nov. 9, 2020, the disclosure of which is incorporated by reference herein in its entirety as part of the present application.

### TECHNICAL FIELD

The present disclosure belongs to the technical field of sequence data analysis and particularly relates to a method for encoding DNA/RNA sequences.

### BACKGROUND ART

DNA/RNA sequence encoding method is a data processing method which converts DNA/RNA sequences into the numerical data. It plays an important role in solving the problem of identifying and predicting biological epigenetic sites such as DNA methylation sites and RNA methylation sites by using machine learning technology. Whether the DNA/RNA sequence encoding method can effectively extract the numerical features containing strong categorical information from DNA/RNA sequences will determine the performance of the subsequent classification model constructed using the features.

The existing DNA/RNA sequence encoding methods cannot extract the key feature information for effectively identifying the epigenetic sites from the DNA/RNA sequences, therefore, the performance of the subsequent classification model based on the existing DNA/RNA sequence encoding methods is poor. Combining the numerical features obtained by multiple DNA/RNA sequence encoding methods to get the high-dimensional numerical feature vector containing rich identification information can solve the shortcomings of constructing classification model by using a single DNA/RNA sequence encoding method, but it will lead to the high redundancy of the combined high-dimensional numerical features and waste of computing resources, and the improvement on the performance of the model is limited. Therefore, how to encode DNA/RNA sequences into numerical features containing key information while with low redundancy between features for effectively identifying epigenetic sites is the key issue to solve the problem of identification and prediction of biological epigenetic sites, and it is also the research hotspot in the art at present.

### SUMMARY OF THE INVENTION

The technical problem to be solved by the present disclosure is to overcome the aforementioned defects of the prior art, and to provide a method for encoding DNA/RNA sequences based on bidirectional trinucleotide position-specific propensities and pointwise joint mutual information, which can extract the features with strong categorical information, low redundancy between features and high accuracy of the subsequently constructed model.

The technical scheme used for solving the technical problems comprises the following steps:

(1) constructing a nucleotide position-specific propensity matrix of DNA/RNA sequences;

giving a dataset D of DNA/RNA sequences, the dataset consists of a positive dataset and a negative dataset, that is, D=D⁺∪D⁻;

determining a nucleotide position-specific propensity matrix M_{S}⁺ for the positive dataset D⁺ according to the following formula:

$${M}_{s}^{+} = [ \begin{matrix}{f}_{A , 1}^{+} & {f}_{A , 2}^{+} & L & {f}_{A , i}^{+} \\ {f}_{C , 1}^{+} & {f}_{C , 2}^{+} & L & {f}_{C , i}^{+} \\ {f}_{G , 1}^{+} & {f}_{G , 2}^{+} & L & {f}_{G , i}^{+} \\ {f}_{X , 1}^{+} & {f}_{X , 2}^{+} & L & {f}_{X , i}^{+}\end{matrix} ]$$

wherein, A, C, G and X are 4 types of nucleotides of DNA/RNA, and X represents nucleotide T in DNA, and U in RNA, and i represents a position of a nucleotide, 1≤i≤l, i is a finite positive integer, and l is a length of a DNA/RNA sequence; the l is an odd number. f_{A,i}⁺, f_{C,i}⁺, f_{G,i}⁺ and f_{X,i}⁺ are occurrence frequencies of nucleotides A, C, G and X at position i in positive dataset D⁺, respectively.

Determining a nucleotide position-specific propensity matrix M_{S}⁻ of the negative dataset D⁻ according to the following formula:

$${M}_{s}^{-} = [ \begin{matrix}{f}_{A , 1}^{-} & {f}_{A , 2}^{-} & L & {f}_{A , i}^{-} \\ {f}_{C , 1}^{-} & {f}_{C , 2}^{-} & L & {f}_{C , i}^{-} \\ {f}_{G , 1}^{-} & {f}_{G , 2}^{-} & L & {f}_{G , i}^{-} \\ {f}_{X , 1}^{-} & {f}_{X , 2}^{-} & L & {f}_{X , i}^{-}\end{matrix} ]$$

wherein f_{A,i}⁻, f_{C,i}⁻, f_{G,i}⁻ and f_{X,i}⁻ are occurrence frequencies of nucleotides A, C, G and X at position i in negative dataset D⁻, respectively.

(2) Constructing a bidirectional dinucleotide position-specific propensity matrix of DNA/RNA sequences;

determining a forward dinucleotide position-specific propensity matrix

$${{M}^{\text{?}}}_{d}$$

$$\text{?} \text{indicates text missing or illegible when filed}$$

for the positive dataset D⁺ according to the following formula:

$${{M}^{uur}}_{d}^{+} = [ \begin{matrix}{{f}^{ur}}_{AA , 1}^{+} & {{f}^{ur}}_{AA , 2}^{+} & L & {{f}^{ur}}_{AA , j}^{+} \\ {{f}^{ur}}_{AC , 1}^{+} & {{f}^{ur}}_{AC , 2}^{+} & L & {{f}^{ur}}_{AC , j}^{+} \\ M & M & O & M \\ {{f}^{ur}}_{XX , 1}^{+} & {{f}^{ur}}_{XX , 2}^{+} & L & {{f}^{ur}}_{XX , j}^{+}\end{matrix} ]$$

wherein, AA, AC, . . . , and XX are 16 types of dinucleotides formed by the 4 types of nucleotides A, C, G, and X of DNA/RNA, j represents position of dinucleotide, 2≤j≤l−1, j is a finite positive integer, l is a length of a DNA/RNA sequence,

$${{f}^{ur}}_{AA , j}^{+} , {{f}^{ur}}_{AC , j}^{+} , … , and {{f}^{ur}}_{XX , j}^{+}$$

are occurrence frequencies of dinucleotides of AA, AC, . . . , and XX in the positive dataset D⁺, wherein a first nucleotide of a dinucleotide is at position j and a second nucleotide is at position J+1, respectively.

Determining a backward dinucleotide position-specific propensity matrix

$${{M}^{sun}}_{d}^{+}$$

for the positive dataset D⁺ according to the following formula:

$${{M}^{sun}}_{d}^{+} = [ \begin{matrix}{{f}^{su}}_{AA , 2}^{+} & {{f}^{su}}_{AA , 3}^{+} & L & {{f}^{su}}_{AA , j}^{+} \\ {{f}^{su}}_{AC , 2}^{+} & {{f}^{su}}_{AC , 3}^{+} & L & {{f}^{su}}_{AC , j}^{+} \\ M & M & O & M \\ {{f}^{su}}_{XX , 2}^{+} & {{f}^{su}}_{XX , 3}^{+} & L & {{f}^{su}}_{XX , j}^{+}\end{matrix} ]$$

wherein,

$${{f}^{su}}_{AA , j}^{+} , {{f}^{su}}_{AC , j}^{+} , … , and {{f}^{su}}_{XX , j}^{+}$$

are occurrence frequencies of dinucleotides of AA, AC, . . . , and XX in positive dataset D⁺, respectively, wherein, a first nucleotide of a dinucleotide is at position j and a second nucleotide is at position j−1, respectively.

Determining a forward dinucleotide position-specific propensity matrix

$${{M}^{uur}}_{d}^{-}$$

for the negative dataset D⁻ according to the following formula:

$${{M}^{uur}}_{d}^{-} = [ \begin{matrix}{{f}^{ur}}_{AA , 2}^{-} & {{f}^{ur}}_{AA , 3}^{-} & L & {{f}^{ur}}_{AA , j}^{-} \\ {{f}^{ur}}_{AC , 2}^{-} & {{f}^{ur}}_{AC , 3}^{-} & L & {{f}^{ur}}_{AC , j}^{-} \\ M & M & O & M \\ {{f}^{ur}}_{XX , 2}^{-} & {{f}^{ur}}_{XX , 3}^{-} & L & {{f}^{ur}}_{XX , j}^{-}\end{matrix} ]$$

wherein

$${{f}^{ur}}_{AA , j}^{-} , {{f}^{ur}}_{AC , j}^{-} , … , and {{f}^{ur}}_{XX , j}^{-}$$

are occurrence frequencies of dinucleotides AA, AC, . . . , and XX in negative dataset D⁻, respectively, wherein, a first nucleotide of a dinucleotide is at position j and a second nucleotide is at position j+1, respectively.

Determining a backward dinucleotide position-specific propensity matrix

$${{M}^{\text{?}}}_{d}$$

$$\text{?} \text{indicates text missing or illegible when filed}$$

for the negative dataset according to the following formula:

$${{M}^{sun}}_{d}^{-} = [ \begin{matrix}{{f}^{su}}_{AA , 2}^{-} & {{f}^{su}}_{AA , 3}^{-} & L & {{f}^{su}}_{AA , j}^{-} \\ {{f}^{su}}_{AC , 2}^{-} & {{f}^{su}}_{AC , 3}^{-} & L & {{f}^{su}}_{AC , j}^{-} \\ M & M & O & M \\ {{f}^{su}}_{XX , 2}^{-} & {{f}^{su}}_{XX , 3}^{-} & L & {{f}^{su}}_{XX , j}^{-}\end{matrix} ]$$

wherein,

$${{f}^{su}}_{AA , j}^{-} , {{f}^{su}}_{AC , j}^{-} , … , and {{f}^{su}}_{XX , j}^{-}$$

are occurrence frequencies of dinucleotides AA, AC, . . . , and XX of negative dataset D⁻, respectively, wherein a first nucleotide of a dinucleotide is at position j and a second nucleotide is at position j−1, respectively.

(3) Constructing a bidirectional trinucleotide position-specific propensity matrix of DNA/RNA sequences

determining a forward trinucleotide position-specific propensity matrix

$${{M}^{uur}}_{t}^{+}$$

for the positive dataset D⁺ according to the following formula:

$${{M}^{uur}}_{t}^{+} = [ \begin{matrix}{{f}^{ur}}_{AAA , β + 3}^{+} & {{f}^{ur}}_{AAA , β + 4}^{+} & L & {{f}^{ur}}_{AAA , k}^{+} \\ {{f}^{ur}}_{AAC , β + 3}^{+} & {{f}^{ur}}_{AAC , β + 4}^{+} & L & {{f}^{ur}}_{AAC , k}^{+} \\ M & M & O & M \\ {{f}^{ur}}_{XXX , β + 3}^{+} & {{f}^{ur}}_{XXX , β + 4}^{+} & L & {{f}^{ur}}_{XXX , k}^{+}\end{matrix} ]$$

wherein AAA, AAC, . . . , XXX are 64 types of trinucleotides formed by 4 types of nucleotides A, C, G, and X of DNA/RNA, β represents a distance between the nucleotide at position k and its forward adjacent dinucleotide, 0≤β≤(l−5)/2, and β is a positive integer, l is a length of a DNA/RNA sequence, k is a finite positive integer, k represents a position of a first nucleotide of the forward trinucleotide, β+3≤k≤l−β−2, then a second nucleotide is at position k+β+1 and a third at k+β+2.

$${{f}^{ur}}_{AAA , k}^{+} , {{f}^{ur}}_{AAC , k}^{+} , … , and {{f}^{ur}}_{XXX , k}^{+}$$

are occurrence frequencies of trinucleotides of AAA, AAC, . . . , and XXX of positive dataset D⁺.

Determining a backward trinucleotide position-specific propensity matrix

$${{M}^{sun}}_{t}^{+}$$

for the positive dataset D⁺ according to the following formula:

$$\begin{matrix}{su}_{+} \\ {M}_{t}\end{matrix} = [ \begin{matrix}\begin{matrix}{su}_{+} \\ {f}_{A A A , β + 3}\end{matrix} & \begin{matrix}{su}_{+} \\ {f}_{A A A , β + 4}\end{matrix} & L & \begin{matrix}{su}_{+} \\ {f}_{AAA , k}\end{matrix} \\ \begin{matrix}{su}_{+} \\ {f}_{A A C , β + 3}\end{matrix} & \begin{matrix}{su}_{+} \\ {f}_{A A C , β + 4}\end{matrix} & L & \begin{matrix}{su}_{+} \\ {f}_{AAC , k}\end{matrix} \\ M & M & O & M \\ \begin{matrix}{su}_{+} \\ {f}_{X X X , β + 3}\end{matrix} & \begin{matrix}{su}_{+} \\ {f}_{X X X , β + 4}\end{matrix} & L & \begin{matrix}{su}_{+} \\ {f}_{xxx , k}\end{matrix}\end{matrix} ]$$

wherein,

$$\begin{matrix}{su}_{+} \\ {f}_{A A A , k} ,\end{matrix} \begin{matrix}{su}_{+} \\ {f}_{AAC , k} ,\end{matrix} \begin{matrix} \\ … ,\end{matrix} \begin{matrix} \\ and\end{matrix} \begin{matrix}{su}_{+} \\ {f}_{XXX , k}\end{matrix}$$

are occurrence frequencies of trinucleotides AAA, AAC, . . . , and XXX of positive dataset D⁺, respectively, wherein a first, second, and a third nucleotide of the backward trinucleotide are at positions k, k−β−1, and k−β−2, respectively, of sequences.

Determining a forward trinucleotide position-specific propensity matrix

$$\begin{matrix}u {\text{?}}_{-} \\ {M}_{t}\end{matrix} \\ \text{?} \text{indicates text missing or illegible when filed}$$

for the negative dataset D⁻ according to the following formula:

$$\begin{matrix}u {\text{?}}_{-} \\ {M}_{t}\end{matrix} = [ \begin{matrix}\begin{matrix}{ur}_{-} \\ {f}_{A A A , β + 3}\end{matrix} & \begin{matrix}{ur}_{-} \\ {f}_{A A A , β + 4}\end{matrix} & L & \begin{matrix}{ur}_{-} \\ {f}_{AAA , k}\end{matrix} \\ \begin{matrix}{ur}_{-} \\ {f}_{A A C , β + 3}\end{matrix} & \begin{matrix}{ur}_{-} \\ {f}_{A A C , β + 4}\end{matrix} & L & \begin{matrix}{ur}_{-} \\ {f}_{AAC , k}\end{matrix} \\ M & M & O & M \\ \begin{matrix}{ur}_{-} \\ {f}_{X X X , β + 3}\end{matrix} & \begin{matrix}{ur}_{-} \\ {f}_{X X X , β + 4}\end{matrix} & L & \begin{matrix}{ur}_{-} \\ {f}_{xxx , k}\end{matrix}\end{matrix} ] \\ \text{?} \text{indicates text missing or illegible when filed}$$

wherein,

$$\begin{matrix}{ur}_{-} \\ {f}_{A A A , k} ,\end{matrix} \begin{matrix}{ur}_{-} \\ {f}_{AAC , k} ,\end{matrix} \begin{matrix} \\ … ,\end{matrix} \begin{matrix} \\ and\end{matrix} \begin{matrix}{ur}_{-} \\ {f}_{XXX , k}\end{matrix}$$

are occurrence frequencies of trinucleotides of AAA, AAC, . . . , and XXX of negative dataset D⁻, respectively, wherein a first, second, and third nucleotide of the above forward trinucleotides are at positions k, k+β+1, and k+β+2, respectively, of the sequences.

Determining a backward trinucleotide position-specific propensity matrix

$${{M}^{\text{?}}}_{t}$$

$$\text{?} \text{indicates text missing or illegible when filed}$$

for the negative dataset D⁻ according to the following formula:

$$\begin{matrix}{su}_{-} \\ {M}_{t}\end{matrix} = [ \begin{matrix}\begin{matrix}{su}_{-} \\ {f}_{A A A , β + 3}\end{matrix} & \begin{matrix}{su}_{-} \\ {f}_{A A A , β + 4}\end{matrix} & L & \begin{matrix}{su}_{-} \\ {f}_{AAA , k}\end{matrix} \\ \begin{matrix}{su}_{-} \\ {f}_{A A C , β + 3}\end{matrix} & \begin{matrix}{su}_{-} \\ {f}_{A A C , β + 4}\end{matrix} & L & \begin{matrix}{su}_{-} \\ {f}_{AAC , k}\end{matrix} \\ M & M & O & M \\ \begin{matrix}{su}_{-} \\ {f}_{X X X , β + 3}\end{matrix} & \begin{matrix}{su}_{-} \\ {f}_{X X X , β + 4}\end{matrix} & L & \begin{matrix}{su}_{-} \\ {f}_{xxx , k}\end{matrix}\end{matrix} ]$$

wherein,

$$\begin{matrix}{su}_{-} \\ {f}_{A A A , k} ,\end{matrix} \begin{matrix}{su}_{-} \\ {f}_{AAC , k} ,\end{matrix} \begin{matrix} \\ … ,\end{matrix} \begin{matrix} \\ and\end{matrix} \begin{matrix}{su}_{-} \\ {f}_{XXX , k}\end{matrix}$$

are occurrence frequencies of trinucleotides AAA, AAC, . . . , and XXX of negative dataset D⁻, respectively, wherein a first, second and third nucleotide of the above backward trinucleotides are at positions k, k−β−1, and k−β−2, respectively, of all sequences.

(4) Determining a value of pointwise joint mutual information of the nucleotides of DNA/RNA sequences

(4.1) Determining a value

$$\begin{matrix}{r}_{+} \\ {v}_{k}\end{matrix}$$

of forward pointwise joint mutual information of nucleotides of DNA/RNA sequences to be encoded in the positive dataset D⁺ according to the following formula:

$$\begin{matrix}{r}_{+} \\ {v}_{k}\end{matrix} = log \frac{\begin{matrix}{ur}_{+} \\ {f}_{xyz , k}^{ur}\end{matrix}}{\begin{matrix}{ur}_{+} \\ {f}_{x , k}^{+} {f}_{y z , k + β + 1}^{ur}\end{matrix}}$$

wherein, x is a nucleotide at position k, x∈{A, C, G, X},

$$\begin{matrix}u \\ y\end{matrix}$$

is a nucleotide at position k+β+1,

$$\begin{matrix}u \\ y ∈ \{ A , C , G , X \} ,\end{matrix} \begin{matrix}1 \\ z\end{matrix}$$

is a nucleotide at position k+β+2,

$$\begin{matrix}1 \\ z ∈ \{ A , C , G , X \} ,\end{matrix} \begin{matrix} \\ and\end{matrix} \begin{matrix}{ur}_{+} \\ {f}_{x y z , k}^{ur}\end{matrix}$$

is an occurrence frequency of trinucleotide

$${xyz}^{\text{?}}$$

$$\text{?} \text{indicates text missing or illegible when filed}$$

in positive dataset D⁺,

$$\begin{matrix}{ur}_{+} \\ {f}_{yz , k + β + 1}^{ur}\end{matrix}$$

is an occurrence frequency of dinucleotide

$${yz}^{\text{?}}$$

$$\text{?} \text{indicates text missing or illegible when filed}$$

of all sequence samples of positive dataset D⁺, and f_{x,k}⁺ is an occurrence frequency of nucleotide x at position k of all sequence samples of positive dataset D⁺.

Determining a value

$$\begin{matrix}{s}_{+} \\ {v}_{k}\end{matrix}$$

of backward pointwise joint mutual information of nucleotides of DNA/RNA sequences to be encoded in the positive dataset D⁺ according to the following formula:

$$\begin{matrix}{s}_{+} \\ {v}_{k}\end{matrix} = log \frac{\begin{matrix}{su}_{+} \\ {f}_{xyz , k} \text{?} s\end{matrix}}{\begin{matrix}{su}_{+} \\ {f}_{x , k}^{+} {f}_{yz , k - β - 1} \text{?}\end{matrix}} \\ \text{?} \text{indicates text missing or illegible when filed}$$

wherein, x is a nucleotide at position k, xε{A, C, G, X},

$${y}^{\text{?}}$$

$$\text{?} \text{indicates text missing or illegible when filed}$$

is a nucleotide at position k−β−1,

$${y}^{\text{?}} ∈ \{ A , C , G , X \} , {z}^{s}$$

$$\text{?} \text{indicates text missing or illegible when filed}$$

is a nucleotide at position k−β−2,

$${z}^{s} ∈ \{ A , C , G , X \} , and {{f}^{{su}_{+}}}_{xyz , k}^{\text{?}}$$

$$\text{?} \text{indicates text missing or illegible when filed}$$

represents an occurrence frequency of trinucleotide

$${xyz}^{\text{?}}$$

$$\text{?} \text{indicates text missing or illegible when filed}$$

of all sequences in positive dataset D⁺,

$${{f}^{{su}_{+}}}_{yz , k - β - 1}^{\text{?}}$$

$$\text{?} \text{indicates text missing or illegible when filed}$$

represents an occurrence frequency of dinucleotide

$${yz}^{\text{?}}$$

$$\text{?} \text{indicates text missing or illegible when filed}$$

of all sequences in positive dataset D⁺.

The encoding value vₖ⁺ of pointwise joint mutual information in the positive dataset D⁺ of a nucleotide at position k of DNA/RNA sequences to be encoded is defined as an average value of the value

$${{v}_{k}}^{{r}_{+}}$$

of forward pointwise joint mutual information and the value

$${{v}_{k}}^{{s}_{+}}$$

of backward pointwise joint mutual information. The DNA/RNA sequence with length l is encoded into a pointwise mutual information feature vector V⁺ with length of l−2β−4:

$${V}^{+} = [ {v}_{β + 3}^{+} , {v}_{β + 4}^{+} , L , {v}_{k}^{+} ]$$

$${v}_{k}^{+} = \frac{{{v}_{k}}^{{r}_{+}} + {{v}_{k}}^{{s}_{+}}}{2}$$

(4.2) Determining a value

$${{v}_{k}}^{{r}_{-}}$$

of forward pointwise joint mutual information of nucleotides of DNA/RNA sequences to be encoded in the negative dataset D⁻ according to the following formula:

$${{v}_{k}}^{{r}_{-}} = log \frac{{{f}^{\text{?}}}_{xyz , k}^{\text{?}}}{{f}_{x , k}^{-} {{f}^{\text{?}}}_{yz , k + β + 1}^{\text{?}}}$$

$$\text{?} \text{indicates text missing or illegible when filed}$$

Wherein,

$${{f}^{\text{?}}}_{xyz , k}^{\text{?}}$$

$$\text{?} \text{indicates text missing or illegible when filed}$$

represents an occurrence frequency of trinucleotide

$$\begin{matrix}\text{?} 1 \\ xyz\end{matrix} \\ \text{?} \text{indicates text missing or illegible when filed}$$

in negative dataset D⁻, and x,

$${y}^{\text{?}} , and {z}^{\text{?}}$$

$$\text{?} \text{indicates text missing or illegible when filed}$$

are nucleotides at positions k, k+β+1 and k+β+2, respectively.

$${{f}^{\text{?}}}_{yz , k + β + 1}^{\text{?}}$$

$$\text{?} \text{indicates text missing or illegible when filed}$$

is an occurrence frequency of dinucleotide

$$\begin{matrix}\text{?} 1 \\ yz\end{matrix} \\ \text{?} \text{indicates text missing or illegible when filed}$$

in negative dataset D⁻, and f_{x,k}⁻ is an occurrence frequency of nucleotide x in negative dataset D⁻.

Determining a value

$${{v}_{k}}^{{s}_{-}}$$

of backward pointwise joint mutual information of nucleotides of DNA/RNA sequences to be encoded in the negative dataset D⁻ according to the following formula:

$$\begin{matrix}{s}_{-} \\ {v}_{k}\end{matrix} = log \frac{\begin{matrix}{su}_{-} \\ {f}_{xyz , k} \text{?} s\end{matrix}}{\begin{matrix}{su}_{-} \\ {f}_{x , k}^{-} {f}_{yz , k - β - 1} \text{?} s\end{matrix}} \\ \text{?} \text{indicates text missing or illegible when filed}$$

wherein,

$$\begin{matrix}{su}_{-} \\ {f}_{xyz , k} \text{?} s\end{matrix} \\ \text{?} \text{indicates text missing or illegible when filed}$$

is an occurrence frequency of trinucleotide

$$\begin{matrix}\text{?} s \\ xyz\end{matrix} \\ \text{?} \text{indicates text missing or illegible when filed}$$

of all sequences of negative dataset

$${D}^{-} \cdot \begin{matrix}\text{?} s \\ x , y , z\end{matrix} \\ \text{?} \text{indicates text missing or illegible when filed}$$

are nucleotides at positions k, k−β−1 and k−β−2, respectively.

$$\begin{matrix}{su}_{-} \\ {f}_{yz , k - β - 1} \text{?} s\end{matrix} \\ \text{?} \text{indicates text missing or illegible when filed}$$

is an occurrence frequency of dinucleotide

$$\begin{matrix}\text{?} s \\ yz\end{matrix} \\ \text{?} \text{indicates text missing or illegible when filed}$$

of all sequences of negative dataset D⁻.

The encoding value vₖ⁻ of pointwise joint mutual information of a nucleotide at position k of DNA/RNA sequences to be encoded in the negative dataset D⁻ is defined as an average of the value

$$\begin{matrix}{r}_{-} \\ {v}_{k}\end{matrix}$$

of forward pointwise joint mutual information and the value

$$\begin{matrix}{s}_{-} \\ {v}_{k}\end{matrix}$$

of backward pointwise joint mutual information, and a DNA/RNA sequence with length l is encoded into a pointwise mutual information feature vector V⁻ with a length of l−2,β−4:

$${V}^{-} = [ {v}_{β + 3}^{-} , {v}_{β + 4}^{-} , L , {v}_{k}^{-} ] \\ {v}_{k}^{-} = \frac{\begin{matrix}{r}_{-} {s}_{-} \\ {v}_{k} + {v}_{k}\end{matrix}}{2}$$

(4.3) Determining a feature vector V of a DNA/RNA sequence to be encoded with a given length l by corresponding element of vector V⁺ minus that of V⁻:

V=[V_{β+3}, V_{β+4}, . . . , Vₖ]

Vₖ=vₖ⁺−vₖ⁻

(5) Concatenating Features

When the value of parameter β is 0, the feature vector V(0) is [V₃, V₄, V₅, . . . , Vₗ₋₃, Vₗ₋₂], and the number of elements is l−4. When the value of β is 1, the feature vector V(1) is [V₄, V₅, V₆, . . . , Vₗ₋₄, Vₗ₋₃], and the number of elements is l−6, . . . , and when the value of β is (l−7)/2, the feature vector V((l−7)/2) is [V_{(l-1)/2}, V_{(l+1)/2}, V_{(l+3)/2}], the number of elements is 3. When the value β is (l−5)/2, the feature vector V((l−5)/2) is [V_{(l+1)/2}], and the number of elements is 1. Concatenating the feature vectors determined by different values of parameter β into a high-dimensional feature vector [V(0), V(1), . . . , V((l−7)/2), V((l−5)/2)] with (l−3)²/4 elements.

(6) Encoding DNA/RNA Sequences

Encoding the DNA/RNA sequence dataset D into a numerical dataset D′ by performing the above step (1)-step (5),

$${D}^{′} ∈ {R}^{s \times \frac{{( l - 3 )}^{2}}{4}} ,$$

where s is a number of samples in the numerical dataset D′, that is, the number of the DNA/RNA sequences in dataset D. The (l−3)²/4 is a feature number of the numerical dataset D′.

In the present disclosure, a bidirectional dinucleotide position-specific propensity and a trinucleotide position-specific propensity are proposed based on nucleotide position-specific propensities, and a pointwise joint mutual information is proposed based on nucleotide position-specific propensity matrix and bidirectional dinucleotide position-specific propensity matrix and bidirectional trinucleotide position-specific propensity matrix, then an encoding method is proposed for representing DNA/RNA sequences by using pointwise joint mutual information and nucleotide position-specific propensity matrix and bidirectional dinucleotide position-specific propensity matrix and bidirectional trinucleotide position-specific propensity matrix of positive and negative datasets of DNA/RNA sequences, and DNA/RNA sequences are encoded into numerical feature samples. In order to extract more trinucleotide position information from DNA/RNA sequences, the parameter β is introduced into the process of constructing the bidirectional trinucleotide position-specific propensity matrix to represent the distance between the current nucleotide and its forward or backward adjacent dinucleotide, and the numerical feature vectors obtained from different values of β are concatenated, so as to obtain a high-dimensional numerical feature vector with global and local categorical information and low redundancy between features. The simulation comparative experiments are carried out by using the encoding method provided by the present disclosure and the existing seven encoding methods, and the experimental results show that the accuracy, sensitivity, specificity, MCC (Mathew's correlation coefficient), AUROC (Area under the receiver operating characteristic curve) and AUPRC (Area under the precision recall curve) of the support vector machine model constructed based on the encoding method provided by the present disclosure for identifying the DNA N⁴-methylcytosine (4mC) sites in the Caenorhabditis elegans DNA sequences are 0.987, 0.991, 0.983, 0.974, 0.999 and 0.999, respectively, which are much higher than those of the other seven compared encoding methods; the accuracy, sensitivity, specificity, MCC, AUROC and AUPRC of the support vector machine model constructed based on the encoding method provided by the present disclosure for identifying the RNA N⁶-methyladenosine (m⁶A) sites in the Saccharomyces cerevisiae RNA sequences are 0.995, 0.996, 0.994, 0.990, 1 and 1, respectively, which are much higher than those of the other seven compared encoding methods.

### BRIEF DESCRIPTION OF THE DRAWINGS

FIG. 1 is the flowchart of the method of the present disclosure.

FIG. 2 shows the AUROC curves of the support vector machine models for identifying the DNA N⁴-methylcytosine sites in the DNA sequence of Caenorhabditis elegans based on the encoding method provided by the present disclosure and seven encoding methods, respectively.

FIG. 3 shows the AUPRC curves of the support vector machine models for identifying the DNA N⁴-methylcytosine sites in the DNA sequence of Caenorhabditis elegans based on the encoding method provided by the present disclosure and seven encoding methods, respectively.

FIG. 4 shows the AUROC curves of the support vector machine models for identifying the RNA N⁶-methyladenosine sites in the Saccharomyces cerevisiae RNA sequences based on the encoding method provided by the present disclosure and seven encoding methods, respectively.

FIG. 5 shows the AUPRC curves of the support vector machine models for identifying the RNA N⁶-methyladenosine sites in the Saccharomyces cerevisiae RNA sequences based on the encoding method provided by the present disclosure and seven encoding methods, respectively.

### DETAILED DESCRIPTION OF THE EMBODIMENTS

The technical schemes provided by the present disclosure will be described in detail below with reference to the figures and examples, but they should not be understood as any limitation to the scope of the present disclosure.

### Example 1

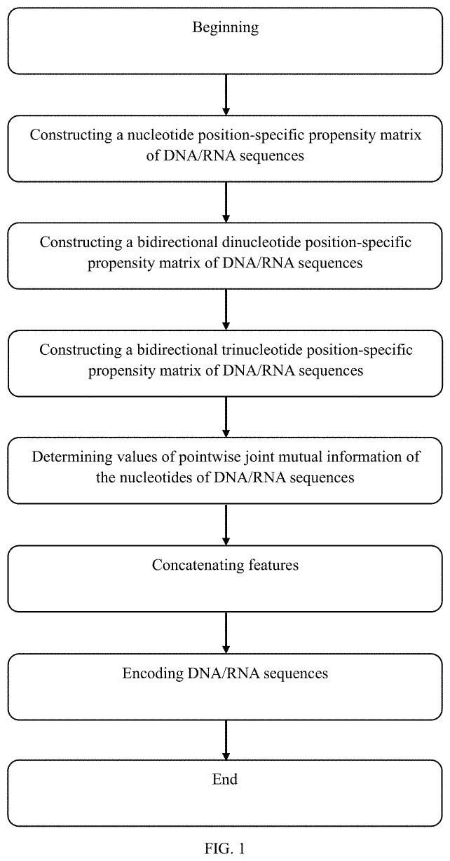

The DNA N⁴-methylcytosine (4mC) dataset of the Caenorhabditis elegans×DNA sequences recorded in the literature “iDNA4mC: identifying DNA N⁴-methylcytosine sites based on nucleotide chemical properties” was taken as an example. The dataset consisted of 3108 DNA sequences, of which, the number of sequences in positive dataset, i.e., the number of actual N⁴-methylcytosine samples, was 1554, the number of sequences in negative dataset, i.e., the number of non-N⁴-methylcytosine samples, was 1554, and the length l of each sequence was 41. The method for encoding the DNA sequences based on the bidirectional trinucleotide position-specific propensities and pointwise joint mutual information of this present example comprises the following steps (reference FIG. 1):

(1) a nucleotide position-specific propensity matrix of DNA sequences was constructed;

A dataset D of DNA sequences was given, and it consisted of a positive dataset D⁺ and a negative dataset D⁻, i.e. D=D⁺D∪D⁻;

the nucleotide position-specific propensity matrix M_{S}⁺ for the positive dataset D⁺ was determined according to the following formula:

$${M}_{s}^{+} = [ \begin{matrix}{f}_{A , 1}^{+} & {f}_{A , 2}^{+} & L & {f}_{A , i}^{+} \\ {f}_{C , 1}^{+} & {f}_{C , 2}^{+} & L & {f}_{C , i}^{+} \\ {f}_{G , 1}^{+} & {f}_{G , 2}^{+} & L & {f}_{G , i}^{+} \\ {f}_{T , 1}^{+} & {f}_{T , 2}^{+} & L & {f}_{T , i}^{+}\end{matrix} ]$$

where, A, C, G and T were the 4 types of nucleotides of DNA sequences, i represents the position of a nucleotide, 1≤i≤l, and i was a positive integer, and l was the length of a DNA sequence, and it was an odd number, the value of l in this example was 41, f_{A,i}⁺, f_{C,i}⁺, f_{G,i}⁺ and f_{T,i}⁺ were occurrence frequencies of nucleotides A, C, G and T at position i of all sequences of positive dataset D⁺, respectively;

The nucleotide position-specific propensity matrix M_{S}⁻ of the negative dataset D⁻ was determined according to the following formula:

$${M}_{s}^{-} = [ \begin{matrix}{f}_{A , 1}^{-} & {f}_{A , 2}^{-} & L & {f}_{A , i}^{-} \\ {f}_{C , 1}^{-} & {f}_{C , 2}^{-} & L & {f}_{C , i}^{-} \\ {f}_{G , 1}^{-} & {f}_{G , 2}^{-} & L & {f}_{G , i}^{-} \\ {f}_{T , 1}^{-} & {f}_{T , 2}^{-} & L & {f}_{T , i}^{-}\end{matrix} ]$$

wherein f_{A,i}⁻, f_{C,i}⁻, f_{G,i}⁻ and f_{T,i}⁻ were the occurrence frequencies of nucleotides A, C, G and T at position i of all sequences of negative dataset D⁻, respectively.

(2) A bidirectional dinucleotide position-specific propensity matrix of DNA sequences were constructed;

The forward dinucleotide position-specific propensity matrix

$$\begin{matrix}{uur}_{+} \\ {M}_{d}\end{matrix}$$

for the positive dataset D⁺ was determined according to the following formula:

$$\begin{matrix}u \text{?} \\ {M}_{d}\end{matrix} = [ \begin{matrix}\begin{matrix}u {\text{?}}_{+} \\ {f}_{AA , 1}\end{matrix} & \begin{matrix}u {\text{?}}_{+} \\ {f}_{AA , 2}\end{matrix} & L & \begin{matrix}u {\text{?}}_{+} \\ {f}_{AA , j}\end{matrix} \\ \begin{matrix}u {\text{?}}_{+} \\ {f}_{AC , 1}\end{matrix} & \begin{matrix}u {\text{?}}_{+} \\ {f}_{AC , 2}\end{matrix} & L & \begin{matrix}u {\text{?}}_{+} \\ {f}_{AC , j}\end{matrix} \\ M & M & O & M \\ \begin{matrix}u {\text{?}}_{+} \\ {f}_{TT , 1}\end{matrix} & \begin{matrix}u {\text{?}}_{+} \\ {f}_{T T , 2}\end{matrix} & L & \begin{matrix}u {\text{?}}_{+} \\ {f}_{T {T}_{, j}}\end{matrix}\end{matrix} ] \\ \text{?} \text{indicates text missing or illegible when filed}$$

wherein, AA, AC, . . . , and TT were the 16 types of dinucleotides formed by the 4 types of nucleotides A, C, G, and T of DNA sequences, j represented the position of the dinucleotide, that is the position of the first nucleotide of the dinucleotide, the second nucleotide of the dinucleotide was at position j+1, 2≤j≤l−1, and j was a finite positive integer, 2≤j≤40 in this example,

$$\begin{matrix}{ur}_{+} \\ {f}_{AA , j}\end{matrix} , \begin{matrix}{ur}_{+} \\ {f}_{AC , j}\end{matrix} , … , and \begin{matrix}{ur}_{+} \\ {f}_{TT , j}\end{matrix}$$

were the occurrence frequencies of dinucleotides AA, AC, . . . , and TT of all sequences of positive dataset D⁺, respectively;

The backward dinucleotide position-specific propensity matrix

$$\begin{matrix}{suu}_{+} \\ {M}_{d}\end{matrix}$$

for the positive dataset D⁺ was determined according to the following formula:

$$\begin{matrix}{suu}_{+} \\ {M}_{d}\end{matrix} = [ \begin{matrix}\begin{matrix}{su}_{+} \\ {f}_{AA , 2}\end{matrix} & \begin{matrix}{su}_{+} \\ {f}_{AA , 3}\end{matrix} & L & \begin{matrix}{su}_{+} \\ {f}_{AA , j}\end{matrix} \\ \begin{matrix}{su}_{+} \\ {f}_{AC , 2}\end{matrix} & \begin{matrix}{su}_{+} \\ {f}_{AC , 3}\end{matrix} & L & \begin{matrix}{su}_{+} \\ {f}_{AC , j}\end{matrix} \\ M & M & O & M \\ \begin{matrix}{su}_{+} \\ {f}_{TT , 2}\end{matrix} & \begin{matrix}{su}_{+} \\ {f}_{TT , 3}\end{matrix} & L & \begin{matrix}{su}_{+} \\ {f}_{TT , j}\end{matrix}\end{matrix} ]$$

wherein,

$$\begin{matrix}{su}_{+} \\ {f}_{AA , j}\end{matrix} , \begin{matrix}{su}_{+} \\ {f}_{AC , j}\end{matrix} , … , and \begin{matrix}{su}_{+} \\ {f}_{TT , j}\end{matrix}$$

were the occurrence frequencies of dinucleotides AA, AC, . . . , and TT in positive dataset D⁺, respectively, and the first and second nucleotide of these dinucleotides were at positions j and j−1, respectively;

The forward dinucleotide position-specific propensity matrix

$$\begin{matrix}{uur}_{-} \\ {M}_{d}\end{matrix}$$

for the negative dataset was determined according to the following formula:

$$\begin{matrix}{uur}_{-} \\ {M}_{d}\end{matrix} = [ \begin{matrix}\begin{matrix}{ur}_{-} \\ {f}_{AA , 2}\end{matrix} & \begin{matrix}{ur}_{-} \\ {f}_{AA , 3}\end{matrix} & L & \begin{matrix}{ur}_{-} \\ {f}_{AA , j}\end{matrix} \\ \begin{matrix}{ur}_{-} \\ {f}_{AC , 2}\end{matrix} & \begin{matrix}{ur}_{-} \\ {f}_{AC , 3}\end{matrix} & L & \begin{matrix}{ur}_{-} \\ {f}_{AC , j}\end{matrix} \\ M & M & O & M \\ \begin{matrix}{ur}_{-} \\ {f}_{TT , 2}\end{matrix} & \begin{matrix}{ur}_{-} \\ {f}_{TT , 3}\end{matrix} & L & \begin{matrix}{ur}_{-} \\ {f}_{TT , j}\end{matrix}\end{matrix} ]$$

wherein

$$\begin{matrix}{ur}_{-} \\ {f}_{AA , j}\end{matrix} , \begin{matrix}{ur}_{-} \\ {f}_{AC , j}\end{matrix} , … , and \begin{matrix}{ur}_{-} \\ {f}_{TT , j}\end{matrix}$$

were occurrence frequencies of dinucleotides AA, AC, . . . , and TT of all sequences in negative dataset D⁻, respectively. The first and second nucleotide of these dinucleotides were at positions j and j+1, respectively;

The backward dinucleotide position-specific propensity matrix

$$\begin{matrix}{suu}_{-} \\ {M}_{d}\end{matrix}$$

for the negative dataset D⁻ was determined according to the following formula:

$$\begin{matrix}{suu}_{-} \\ {M}_{d}\end{matrix} = [ \begin{matrix}\begin{matrix}{su}_{-} \\ {f}_{AA , 2}\end{matrix} & \begin{matrix}{su}_{-} \\ {f}_{AA , 3}\end{matrix} & L & \begin{matrix}{su}_{-} \\ {f}_{AA , j}\end{matrix} \\ \begin{matrix}{su}_{-} \\ {f}_{AC , 2}\end{matrix} & \begin{matrix}{su}_{-} \\ {f}_{AC , 3}\end{matrix} & L & \begin{matrix}{su}_{-} \\ {f}_{AC , j}\end{matrix} \\ M & M & O & M \\ \begin{matrix}{su}_{-} \\ {f}_{TT , 2}\end{matrix} & \begin{matrix}{su}_{-} \\ {f}_{TT , 3}\end{matrix} & L & \begin{matrix}{su}_{-} \\ {f}_{TT , j}\end{matrix}\end{matrix} ]$$

wherein,

$$\begin{matrix}{su}_{-} \\ {f}_{AA , j}\end{matrix} , \begin{matrix}{su}_{-} \\ {f}_{AC , j}\end{matrix} , … , and \begin{matrix}{su}_{-} \\ {f}_{TT , j}\end{matrix}$$

were occurrence frequencies of dinucleotides AA, AC, . . . , and TT of all sequences of negative dataset D⁻, respectively. The first and second nucleotide of these dinucleotides were at positions j and j−1, respectively;

(3) A bidirectional trinucleotide position-specific propensity matrix of DNA sequences was constructed

The forward trinucleotide position-specific propensity matrix

$$\begin{matrix}{uur}_{+} \\ {M}_{t}\end{matrix}$$

for the positive dataset D⁺ was determined according to the following formula:

$$\begin{matrix}{uur}_{+} \\ {M}_{d}\end{matrix} = [ \begin{matrix}\begin{matrix}{ur}_{+} \\ {f}_{AAA , β + 3}\end{matrix} & \begin{matrix}{ur}_{+} \\ {f}_{AAA , β + 4}\end{matrix} & L & \begin{matrix}{ur}_{+} \\ {f}_{AAA , k}\end{matrix} \\ \begin{matrix}{ur}_{+} \\ {f}_{AAC , β + 3}\end{matrix} & \begin{matrix}{ur}_{+} \\ {f}_{AAC , β + 4}\end{matrix} & L & \begin{matrix}{ur}_{+} \\ {f}_{AAC , k}\end{matrix} \\ M & M & O & M \\ \begin{matrix}{ur}_{+} \\ {f}_{TTT , β + 3}\end{matrix} & \begin{matrix}{ur}_{+} \\ {f}_{TTT , β + 4}\end{matrix} & L & \begin{matrix}{ur}_{+} \\ {f}_{TTT , k}\end{matrix}\end{matrix} ]$$

wherein AAA, AAC, . . . , TTT were 64 types of trinucleotides formed by 4 types of nucleotides A, C, G, and T of DNA sequences, β represented the distance between the nucleotide at position k and its forward adjacent dinucleotide, 0≤β≤(l−5)/2, β was a positive integer, 0≤β≤18 in this example, k represented a position of trinucleotide, that is, the position of the first nucleotide of a trinucleotide, β+3≤k≤l−β−2, β+3≤k≤39−β in this example, and k was a positive integer,

$$\begin{matrix}{ur}_{+} \\ {f}_{AAA , k}\end{matrix} , \begin{matrix}{ur}_{+} \\ {f}_{AAC , k}\end{matrix} , … , and \begin{matrix}{ur}_{+} \\ {f}_{TTT , k}\end{matrix}$$

represent the frequencies of trinucleotides AAA, AAC, . . . , or TTT of all sequences in positive dataset D⁺, respectively. The first, second and third nucleotide of these trinucleotides were at positions k, k+β+1, and k+β+2 of the DNA sequences, respectively;

The backward trinucleotide position-specific propensity matrix

$$\begin{matrix}{suu}_{+} \\ {M}_{t}\end{matrix}$$

for the positive dataset D⁺ was determined according to the following formula:

$$\begin{matrix}{su}_{+} \\ {M}_{t}\end{matrix} = [ \begin{matrix}\begin{matrix}{su}_{+} \\ {f}_{AAA , β + 3}\end{matrix} & \begin{matrix}{su}_{+} \\ {f}_{AAA , β + 4}\end{matrix} & L & \begin{matrix}{su}_{+} \\ {f}_{AAA , k}\end{matrix} \\ \begin{matrix}{su}_{+} \\ {f}_{AAC , β + 3}\end{matrix} & \begin{matrix}{su}_{+} \\ {f}_{AAC , β + 4}\end{matrix} & L & \begin{matrix}{su}_{+} \\ {f}_{AAC , k}\end{matrix} \\ M & M & O & M \\ \begin{matrix}{su}_{+} \\ {f}_{TTT , β + 3}\end{matrix} & \begin{matrix}{su}_{+} \\ {f}_{TTT , β + 4}\end{matrix} & L & \begin{matrix}{su}_{+} \\ {f}_{TTT , k}\end{matrix}\end{matrix} ]$$

wherein,

$${{f}^{{su}_{+}}}_{AAA , k} , {{f}^{{su}_{+}}}_{AAC , k} , … , and {{f}^{{su}_{+}}}_{TTT , k}$$

were the occurrence frequencies of trinucleotides AAA, AAC, . . . , and TTT of all sequences of positive dataset D⁺, respectively. The first, second and third nucleotide of these trinucleotides were at positions k, k−β−1, and k−β−2, respectively;

The forward trinucleotide position-specific propensity matrix

$$\begin{matrix}{ur}_{-} \\ {M}_{t}\end{matrix}$$

for the negative dataset D⁻ was determined according to the following formula:

$$\begin{matrix}{ur}_{-} \\ {M}_{t}\end{matrix} = [ \begin{matrix}\begin{matrix}{ur}_{-} \\ {f}_{AAA , β + 3}\end{matrix} & \begin{matrix}{ur}_{-} \\ {f}_{AAA , β + 4}\end{matrix} & L & \begin{matrix}{ur}_{-} \\ {f}_{AAA , k}\end{matrix} \\ \begin{matrix}{ur}_{-} \\ {f}_{AAC , β + 3}\end{matrix} & \begin{matrix}{ur}_{-} \\ {f}_{AAC , β + 4}\end{matrix} & L & \begin{matrix}{ur}_{-} \\ {f}_{AAC , k}\end{matrix} \\ M & M & O & M \\ \begin{matrix}{ur}_{-} \\ {f}_{TTT , β + 3}\end{matrix} & \begin{matrix}{ur}_{-} \\ {f}_{TTT , β + 4}\end{matrix} & L & \begin{matrix}{ur}_{-} \\ {f}_{TTT , k}\end{matrix}\end{matrix} ]$$

wherein,

$$\begin{matrix}{ur}_{-} \\ {f}_{AAA , k}\end{matrix} , \begin{matrix}{ur}_{-} \\ {f}_{AAC , k}\end{matrix} , … , and \begin{matrix}{ur}_{-} \\ {f}_{TTT , k}\end{matrix}$$

were the occurrence frequencies of trinucleotides AAA, AAC, . . . , and TTT of all sequences of negative dataset D⁻, respectively. The first, second and third nucleotide of a trinucleotide were at positions k, k+β+1, and k+β+2, respectively;

The backward trinucleotide position-specific propensity matrix

$$\begin{matrix}{su}_{-} \\ {M}_{t}\end{matrix}$$

for the negative dataset D⁻ was determined according to the following formula:

$$\begin{matrix}{su}_{-} \\ {M}_{t}\end{matrix} = [ \begin{matrix}\begin{matrix}{su}_{-} \\ {f}_{AAA , β + 3}\end{matrix} & \begin{matrix}{su}_{-} \\ {f}_{AAA , β + 4}\end{matrix} & L & \begin{matrix}{su}_{-} \\ {f}_{AAA , k}\end{matrix} \\ \begin{matrix}{su}_{-} \\ {f}_{AAC , β + 3}\end{matrix} & \begin{matrix}{su}_{-} \\ {f}_{AAC , β + 4}\end{matrix} & L & \begin{matrix}{su}_{-} \\ {f}_{AAC , k}\end{matrix} \\ M & M & O & M \\ \begin{matrix}{su}_{-} \\ {f}_{TTT , β + 3}\end{matrix} & \begin{matrix}{su}_{-} \\ {f}_{TTT , β + 4}\end{matrix} & L & \begin{matrix}{su}_{-} \\ {f}_{TTT , k}\end{matrix}\end{matrix} ]$$

wherein,

$$\begin{matrix}{su}_{-} \\ {f}_{AAA , k}\end{matrix} , \begin{matrix}{su}_{-} \\ {f}_{AAC , k}\end{matrix} , … , and \begin{matrix}{su}_{-} \\ {f}_{TTT , k}\end{matrix}$$

were the occurrence frequencies of trinucleotides AAA, AAC, . . . , and TTT of all sequences of negative dataset D⁻, respectively. The first, second and third nucleotide of a trinucleotide were at positions k, k−β−1, and k−β−2, respectively;

(4) A value of the pointwise joint mutual information of the nucleotides of DNA sequences was determined

(4.1) The value of the forward pointwise joint mutual information

$$\begin{matrix}{r}_{+} \\ {v}_{k}\end{matrix}$$

of nucleotides of DNA sequences to be encoded in the positive dataset D⁺ was determined according to the following formula:

$$\begin{matrix}{r}_{+} \\ {v}_{k}\end{matrix} = log \frac{\begin{matrix}{ur}_{+} \\ {f}_{xyz , k}^{ur}\end{matrix}}{\begin{matrix}{ur}_{+} \\ {f}_{x , k}^{+}\end{matrix} {f}_{yz , k + β + 1}^{ur}}$$

wherein, x was the nucleotide at position k, X∈{A, C, G, T},

$$\begin{matrix}u \\ y\end{matrix}$$

was the nucleotide at position k+β+1,

$$\begin{matrix}u \\ y ∈ \{ A , C , G , T ) ,\end{matrix} \begin{matrix}r \\ z\end{matrix}$$

was the nucleotide at position k+β+2,

$$\begin{matrix}r \\ z ∈ \{ A , C , G , T \} ,\end{matrix} \begin{matrix}{ur}_{+} \\ {f}_{xyz , k}^{ur}\end{matrix}$$

represents the occurrence frequency of trinucleotide

$$\begin{matrix}ur \\ xyz\end{matrix}$$

of all sequences of positive dataset D⁺,

$$\begin{matrix}{ur}_{+} \\ {f}_{yz , k + β + 1}^{ur}\end{matrix}$$

was the occurrence frequency of dinucleotide

$$\begin{matrix}ur \\ yz\end{matrix}$$

of all sequences of positive dataset D⁺, and f_{x,k}⁺ was the occurrence frequency of nucleotide x of all sequences of positive dataset D⁺;

The value of the backward pointwise joint mutual information

$$\begin{matrix}{s}_{+} \\ {v}_{k}\end{matrix}$$

of nucleotides of DNA sequences to be encoded in the positive dataset D⁺ was determined according to the following formula:

$$\begin{matrix}{s}_{+} \\ {v}_{k}\end{matrix} = log \frac{\begin{matrix}{su}_{+} \\ \text{?}\end{matrix}}{\begin{matrix}{su}_{+} \\ {f}_{x , k}^{+} \text{?}\end{matrix}}$$

$$\text{?} \text{indicates text missing or illegible when filed}$$

wherein,

$$\begin{matrix}\text{?} \\ y\end{matrix}$$

$$\text{?} \text{indicates text missing or illegible when filed}$$

was the nucleotide at position k−β−1,

$$\begin{matrix}\text{?} \\ y\end{matrix} ∈ \{ A , C , G , T \} , \begin{matrix}s \\ z\end{matrix}$$

$$\text{?} \text{indicates text missing or illegible when filed}$$

was the nucleotide at position k−β−2,

$$\begin{matrix}s \\ z\end{matrix} ∈ \{ A , C , G , T \} b , \begin{matrix}{su}_{+} \\ \text{?}\end{matrix}$$

$$\text{?} \text{indicates text missing or illegible when filed}$$

was the occurrence frequency of trinucleotide

$$\begin{matrix}\text{?} \\ xyz\end{matrix}$$

$$\text{?} \text{indicates text missing or illegible when filed}$$

of all sequences of positive dataset D⁺,

$$\begin{matrix}{su}_{+} \\ \text{?}\end{matrix}$$

$$\text{?} \text{indicates text missing or illegible when filed}$$

was the occurrence frequency of dinucleotide

$$\begin{matrix}sus \\ yz\end{matrix}$$

of all sequences of positive dataset D⁺.

The encoding value vₖ⁺ of pointwise joint mutual information of the nucleotide at position k of a DNA sequence to be encoded in the positive dataset D⁺ was defined as the average of the value

$$\begin{matrix}{r}_{+} \\ {v}_{k}\end{matrix}$$

of forward pointwise joint mutual information and the value

$$\begin{matrix}{s}_{+} \\ {v}_{k}\end{matrix}$$

of backward pointwise joint mutual information, and a DNA sequence with length l was encoded into a pointwise mutual information feature vector V⁺ with l−2β−4 elements:

$${V}^{+} = [ {v}_{β + 3}^{+} , {v}_{β + 4}^{+} , L , {v}_{k}^{+} ]$$

$${v}_{k}^{+} = \frac{\begin{matrix}{r}_{+} \\ {v}_{k}\end{matrix} + \begin{matrix}{s}_{+} \\ {v}_{k}\end{matrix}}{2}$$

The value of l was 41 in this example.

(4.2) The value

$$\begin{matrix}{r}_{-} \\ {v}_{k}\end{matrix}$$

of forward pointwise joint mutual information of nucleotides of a DNA sequence to be encoded in the negative dataset D⁻ was determined according to the following formula:

$$\begin{matrix}{r}_{-} \\ {v}_{k}\end{matrix} = log \frac{\text{?}}{\begin{matrix}\text{?} \\ {f}_{x , k}^{-} \text{?}\end{matrix}}$$

$$\text{?} \text{indicates text missing or illegible when filed}$$

wherein, the nucleotides x,

$$\begin{matrix}\text{?} \\ 2\end{matrix} , and \begin{matrix}\text{?} \\ z\end{matrix}$$

$$\text{?} \text{indicates text missing or illegible when filed}$$

were at positions k, k+β+1 and k+β+2, respectively, and the

$$\text{?}$$

$$\text{?} \text{indicates text missing or illegible when filed}$$

was the occurrence frequency of trinucleotide

$$\text{?} xyz$$

$$\text{?} \text{indicates text missing or illegible when filed}$$

of all sequences of negative dataset D⁻,

$$\text{?} {f}_{yz , k + β + 1}^{ur}$$

$$\text{?} \text{indicates text missing or illegible when filed}$$

was the occurrence frequency of dinucleotide

$$\text{?} yz$$

$$\text{?} \text{indicates text missing or illegible when filed}$$

of all sequences of negative dataset D⁻, and f_{h,k}⁻ was the occurrence frequency of the nucleotide x of all sequences of negative dataset D⁻.

The value

$${{v}_{k}}^{{s}_{-}}$$

of backward pointwise joint mutual information of nucleotides of a DNA sequence to be encoded in the negative dataset D⁻ was determined according to the following formula:

$${{v}_{k}}^{{s}_{-}} = log \frac{{f}_{xyz , k} \text{?}}{{f}_{x , k}^{-} {f}_{yz , k - β - 1} \text{?}}$$

$$\text{?} \text{indicates text missing or illegible when filed}$$

wherein, the nucleotides x,

$$\text{?} y , and {z}^{s}$$

$$\text{?} \text{indicates text missing or illegible when filed}$$

were at positions k, k−β−1 and k−β−2, respectively. The

$${f}_{xyz , k} \text{?}$$

$$\text{?} \text{indicates text missing or illegible when filed}$$

was the occurrence frequency of trinucleotide

$$\text{?} x y z$$

$$\text{?} \text{indicates text missing or illegible when filed}$$

of all sequences of negative dataset D⁻. The

$${f}_{xyz , k - β - 1} \text{?}$$

$$\text{?} \text{indicates text missing or illegible when filed}$$

was the occurrence frequency of dinucleotide

$$\text{?} yz$$

$$\text{?} \text{indicates text missing or illegible when filed}$$

of all sequences of negative dataset D⁻.

The encoding value vₖ⁻ of pointwise joint mutual information of the nucleotide at position k of a DNA sequence to be encoded in the negative dataset D⁻ was defined as an average of the value

$${{v}_{k}}^{{r}_{-}}$$

of forward pointwise joint mutual information and the value

$${{v}_{k}}^{{s}_{-}}$$

of backward pointwise joint mutual information, and a DNA sequence with a length of l was encoded into a pointwise mutual information feature vector V⁻ with a length of l−2β−4:

$${V}^{-} = [ {v}_{β + 3}^{-} , {v}_{β + 4}^{-} , L , {v}_{k}^{-} ] \\ \\ \\ {v}_{k}^{-} = \frac{{{v}_{k}}^{{r}_{-}} + {{v}_{k}}^{{s}_{-}}}{2}$$

The value of l was 41 in this example.

(4.3) The feature vector V of a DNA sequence to be encoded with length l was determined by corresponding element of vector V⁺ minus that of V⁻:

V=[V_{β+3}, V_{β+4}, . . . , Vₖ]

Vₖ=vₖ⁺−vₖ⁻;

(5) Concatenating features

when the value of parameter β was 0, the feature vector V(0) was [V₃, V₄, V₅, . . . , Vₗ₋₃, Vₗ₋₂], and the number of elements was l−4; when the value of β was 1, the feature vector V(1) was [V₄, V₅, V₆, . . . , Vₗ₋₄, Vₗ₋₃], and the number of elements was l−6, . . . , and when the value of β was (l−7)/2, the feature vector V((l−7)/2) was [V(_{l-1)/2}, V_{(l+1)/2}, V_{(l+3)/2}], the number of elements was 3; when the value of β was (l−5)/2, the feature vector V((l−5)/2) was [V_{(l+1)/2}], and the number of elements was 1; the feature vectors determined by different values of the parameter β was concatenated into a high-dimensional feature vector [V(0), V(1), . . . , V((l−7)/2), V((l−5)/2)] with (l−3)²/4 elements, the value of l was 41 in this example.

(6) Encoding the DNA sequences

The DNA sequence dataset D was encoded into a numerical dataset D′ by performing the above step (1)-step (5),

$${D}^{′} ∈ {R}^{s \times \frac{{( l - 3 )}^{2}}{4}} ,$$

where s was a number of samples of the numerical dataset D′, and s was a finite positive integer, the value of s was 3108 in this example, i.e. the number of DNA sequences in this DNA sequence dataset D, and (l−3)²/4 was the feature number of the numerical data set D′. The encoding of DNA sequences was completed.

The DNA sequence encoding method of Example 1 was compared with PSNP (position-specific nucleotide propensities), PSDP (position-specific dinucleotide propensities), KNF (K-nucleotide frequencies), KSNPF (K spaced nucleotide pair frequencies), NPPS (nucleotide pair position specificity), PBE (positional binary encoding) and NCPNC (nucleotide chemical property and nucleotide composition) which are for identifying the DNA N⁴-methylcytosine sites in Caenorhabditis elegans DNA sequences by the performance of the support vector machine models constructed using each encoding method. The average classification accuracy, sensitivity, specificity, MCC (Mathew's Correlation coefficient), AUROC (Area under the receiver operating characteristic curve) and AUPRC (Area under the precision recall curve) of the 10-fold cross-validation method were used to evaluate the experimental results. The experimental method was as follows:

1. The DNA sequences of N⁴-methylcytosine of Caenorhabditis elegans were encoded according to the method of Example 1;

2. Normalizing the dataset

The numerical dataset D′ was normalized by the maximum-minimum method according to the following formula:

$${g}_{m n}^{′} = \frac{{g}_{m , n} - min ( {g}_{n} )}{max ( {g}_{n} ) - min ( {g}_{n} )}$$

where g_{m,n} was the n-th feature value of the m-th sample of the numerical dataset D′, the normalized value of g_{m,n} was g′_{m,n}, max(gₙ) and min(gₙ) represent the maximum and minimum feature values of the n-th column of the numerical dataset D′, 1≤m≤s, l≤n≤(l−1)²/4, m and n were finite positive integers, the value of l in this example was 41, and the value of s was 3108.

3. Partitioning dataset

The normalized numerical dataset D′ was partitioned into 10 folds by using the K-fold cross-validation method (K=10). One fold of which was taken as the test dataset D′_{Te}, and the remaining nine folds were taken as the training dataset D′_{Tr}, till each fold was as test dataset, and there were 10 runs in total. The ratio of the training dataset D′_{Tr} to the test dataset D′_{Te} in each run was 9:1.

4. Training and testing the model

The support vector machine model was trained using the training dataset D′_{Tr}, and the performance of the support vector machine model was tested using the test dataset D′_{Te}.

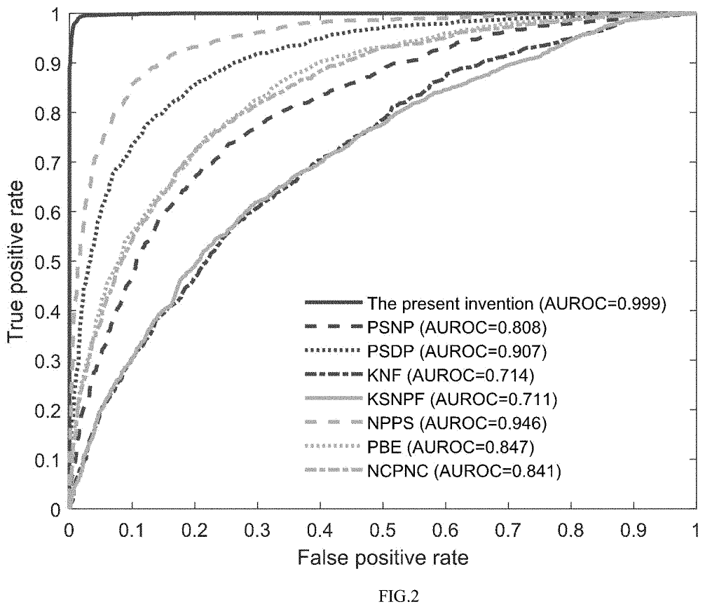

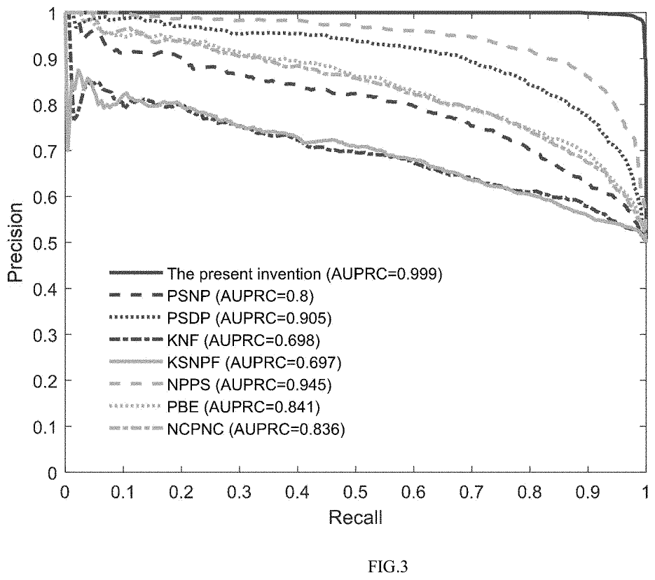

The DNA N⁴-methylcytosine sites in Caenorhabditis elegans DNA sequences were identified by performing the same operation on the seven compared encoding methods according to steps 2-4 of the experimental methods. The experimental results of classification accuracy, sensitivity, specificity and MCC were shown in Table 1, the experimental results of AUROC were shown in FIG. 2, and the experimental results of AUPRC were shown in FIG. 3.

TABLE 1
Comparison of experimental results between the
method of Example 1 and other seven methods

| | Evaluation criterion | | | |
|---|---|---|---|---|
| Encoding method | Accuracy | Sensitivity | Specificity | MCC |
| The present invention | 0.987 | 0.991 | 0.983 | 0.974 |
| PSNP | 0.739 | 0.732 | 0.746 | 0.479 |
| PSDP | 0.827 | 0.820 | 0.833 | 0.653 |
| KNF | 0.653 | 0.656 | 0.651 | 0.307 |
| KSNPF | 0.662 | 0.642 | 0.681 | 0.324 |
| NPPS | 0.877 | 0.880 | 0.873 | 0.754 |
| PBE | 0.763 | 0.775 | 0.750 | 0.526 |
| NCPNC | 0.762 | 0.772 | 0.752 | 0.524 |

As shown in Table 1, the accuracy, sensitivity, specificity and MCC for identifying the DNA N⁴-methylcytosine sites in Caenorhabditis elegans DNA sequences through the support vector machine model constructed based on the DNA sequence encoding method of the present disclosure were 0.987, 0.991, 0.983 and 0.974, respectively, which were much higher than those of the other seven compared encoding methods.

As shown in FIG. 2, the value of AUROC for identifying the DNA N⁴-methylcytosine sites in Caenorhabditis elegans DNA sequences through the support vector machine model constructed based on the DNA sequence encoding method of the present disclosure was 0.999, which was much higher than that of the other seven compared encoding methods.

As shown in FIG. 3, the value of AUPRC for identifying the DNA N⁴-methylcytosine sites in Caenorhabditis elegans DNA sequences through the support vector machine model constructed based on the DNA sequence encoding method of the present disclosure was 0.999, which was much higher than that of the other seven compared encoding methods.

### Example 2

The RNA N⁶-methyladenosine (m⁶A) dataset of the Saccharomyces cerevisiae RNA sequences in the literature “Benchmark data for identifying N⁶-methyladenosine sites in the Saccharomyces cerevisiae genome” was taken as an example. The dataset consisted of 2614 RNA sequences, of which, the number of samples in positive dataset, i.e., the actual number of N⁶-methyladenosine samples, was 1307, the number of samples in negative dataset, i.e., the number of non-N⁶-methyladenosine samples, was 1307, and the length l of each sequence is 51. The method for encoding RNA sequences based on bidirectional trinucleotide position-specific propensities and pointwise joint mutual information of this present example comprises the following steps (reference FIG. 1):

(1) A nucleotide position-specific propensity matrix of RNA sequences was constructed;

A dataset D of RNA sequences was given, and the dataset consisted of a positive dataset D⁺ and a negative dataset D⁻, i.e. D=D⁺∪D⁻;

The nucleotide position-specific propensity matrix M_{S}⁺ for the positive dataset D⁺ was determined according to the following formula:

$${M}_{s}^{+} = [ \begin{matrix}{f}_{A , 1}^{+} & {f}_{A , 2}^{+} & L & {f}_{A , i}^{+} \\ {f}_{C , 1}^{+} & {f}_{C , 2}^{+} & L & {f}_{C , i}^{+} \\ {f}_{G , 1}^{+} & {f}_{G , 2}^{+} & L & {f}_{G , i}^{+} \\ {f}_{U , 1}^{+} & {f}_{U , 2}^{+} & L & {f}_{U , i}^{+}\end{matrix} ]$$

wherein, A, C, G and U were the 4 types of nucleotides of RNA sequences, i represents the position of a nucleotide, 1≤i≤l, and it was a finite positive integer, and l was the length of an RNA sequence, and its value was an odd number, the value of l in this example was 51, f_{A,i}⁺, f_{C,i}⁺, f_{G,i}⁺ and f_{U,i}⁺ were occurrence frequencies of nucleotides A, C, G and U at position i of all sequences of positive dataset D⁺, respectively;

The nucleotide position-specific propensity matrix M_{S}⁻ of the negative dataset D⁻ was determined according to the following formula:

$${M}_{s}^{-} = [ \begin{matrix}{f}_{A , 1}^{-} & {f}_{A , 2}^{-} & L & {f}_{A , i}^{-} \\ {f}_{C , 1}^{-} & {f}_{C , 2}^{-} & L & {f}_{C , i}^{-} \\ {f}_{G , 1}^{-} & {f}_{G , 2}^{-} & L & {f}_{G , i}^{-} \\ {f}_{U , 1}^{-} & {f}_{U , 2}^{-} & L & {f}_{U , i}^{-}\end{matrix} ]$$

wherein f_{A,i}⁻, f_{C,i}⁻, f_{G,i}⁻ and f_{U,i}⁻ were the occurrence frequencies of nucleotides A, C, G and T at position i of all sequences of negative dataset D⁻, respectively.

(2) A bidirectional dinucleotide position-specific propensity matrix of RNA sequences was constructed;

The forward dinucleotide position-specific propensity matrix

$${{M}_{d}}^{{uur}_{+}}$$

for the positive dataset D⁺ was determined according to the following formula:

$${{M}_{d}}^{{uur}_{+}} = [ \begin{matrix}{{f}_{AA , 1}}^{{ur}_{+}} & {{f}_{AA , 2}}^{{ur}_{+}} & L & {{f}_{AA , j}}^{{ur}_{+}} \\ {{f}_{AC , 1}}^{{ur}_{+}} & {{f}_{AC , 2}}^{{ur}_{+}} & L & {{f}_{AC , j}}^{{ur}_{+}} \\ M & M & O & M \\ {{f}_{UU , 1}}^{{ur}_{+}} & {{f}_{UU , 2}}^{{ur}_{+}} & L & {{f}_{UU , j}}^{{ur}_{+}}\end{matrix} ]$$

wherein, AA, AC, . . . , and UU were 16 types of dinucleotides formed by the 4 types of nucleotides A, C, G, and U of RNA sequences, j represents the position of the dinucleotide, i.e., the position of the first nucleotide of the dinucleotides, 2≤j≤l−1, and j was a finite positive integer, 2≤j≤50 in this example,

$${{f}_{AA , j}}^{{ur}_{+}} , {{f}_{AC , j}}^{{ur}_{+}} , … , and {{f}_{UU , j}}^{{ur}_{+}}$$

were the occurrence frequencies of dinucleotides AA, AC, . . . , and UU of all sequences of positive dataset D⁺, respectively, and the first and second nucleotide of the dinucleotides were at positions j and j+1, respectively;

$${{M}_{d}}^{{suu}_{+}}$$

The backward dinucleotide position-specific propensity matrix for the positive dataset D⁺ was determined according to the following formula:

$${{M}_{d}}^{{suu}_{+}} = [ \begin{matrix}{{f}_{AA , 2}}^{{su}_{+}} & {{f}_{AA , 3}}^{{su}_{+}} & L & {{f}_{AA , j}}^{{su}_{+}} \\ {{f}_{AC , 2}}^{{su}_{+}} & {{f}_{AC , 3}}^{{su}_{+}} & L & {{f}_{AC , j}}^{{su}_{+}} \\ M & M & O & M \\ {{f}_{UU , 2}}^{{su}_{+}} & {{f}_{UU , 3}}^{{su}_{+}} & L & {{f}_{UU , j}}^{{su}_{+}}\end{matrix} ]$$

wherein

$${{f}_{AA , j}}^{{su}_{+}} , {{f}_{AC , j}}^{{su}_{+}} , … , and {{f}_{UU , j}}^{{su}_{+}}$$

were the occurrence frequencies of dinucleotides AA, AC, . . . , and UU of all sequences of positive dataset D⁺, respectively. The first and second nucleotide of these dinucleotides were at positions j and j−1, respectively;

The forward dinucleotide position-specific propensity matrix

$${{M}_{d}}^{{uur}_{-}}$$

for the negative dataset D⁻ was determined according to the following formula:

$${{M}_{d}}^{{uur}_{-}} = [ \begin{matrix}{{f}_{AA , 2}}^{{ur}_{-}} & {{f}_{AA , 3}}^{{ur}_{-}} & L & {{f}_{AA , j}}^{{ur}_{-}} \\ {{f}_{AC , 2}}^{{ur}_{-}} & {{f}_{AC , 3}}^{{ur}_{-}} & L & {{f}_{AC , j}}^{{ur}_{-}} \\ M & M & O & M \\ {{f}_{UU , 2}}^{{ur}_{-}} & {{f}_{UU , 3}}^{{ur}_{-}} & L & {{f}_{UU , j}}^{{ur}_{-}}\end{matrix} ]$$

wherein

$${{f}_{AA , j}}^{{ur}_{-}} , {{f}_{AC , j}}^{{ur}_{-}} , … , and {{f}_{UU , j}}^{{ur}_{-}}$$

were the occurrence frequencies of dinucleotides AA, AC, . . . , and UU, whose nucleotides were at positions j and j+1, of all sequences of negative dataset D⁻, respectively;

The backward dinucleotide position-specific propensity matrix

$${{M}_{d}}^{{suu}_{-}}$$

for the negative dataset D⁻ was determined according to the following formula:

$${{M}_{d}}^{{suu}_{-}} = [ \begin{matrix}{{f}_{AA , 2}}^{{su}_{-}} & {{f}_{AA , 3}}^{{su}_{-}} & L & {{f}_{AA , j}}^{{su}_{-}} \\ {{f}_{AC , 2}}^{{su}_{-}} & {{f}_{AC , 3}}^{{su}_{-}} & L & {{f}_{AC , j}}^{{su}_{-}} \\ M & M & O & M \\ {{f}_{UU , 2}}^{{su}_{-}} & {{f}_{UU , 3}}^{{su}_{-}} & L & {{f}_{UU , j}}^{{su}_{-}}\end{matrix} ]$$

wherein,

$${{f}_{AA , j}}^{{su}_{-}} , {{f}_{AC , j}}^{{su}_{-}} , … , and {{f}_{UU , j}}^{{su}_{-}}$$

were the occurrence frequencies of dinucleotides AA, AC, . . . , and UU, whose nucleotides were at positions j and j−1 respectively, of all sequences of negative dataset D⁻, respectively;

(3) A bidirectional trinucleotide position-specific propensity matrix of RNA sequences was constructed

The forward trinucleotide position-specific propensity matrix

$${{M}_{t}}^{{uur}_{+}}$$

for the positive dataset D⁺ was determined according to the following formula:

$$\text{?} {M}_{t} = [ \begin{matrix}{{f}_{A A A , β + 3}}^{{ur}_{+}} & {{f}_{A A A , β + 4}}^{{ur}_{+}} & L & {{f}_{AAA , k}}^{{ur}_{+}} \\ {{f}_{AAC , β + 3}}^{{ur}_{+}} & {{f}_{AAC , β + 4}}^{{ur}_{+}} & L & {{f}_{AAC , k}}^{{ur}_{+}} \\ M & M & O & M \\ {{f}_{UUU , β + 3}}^{{ur}_{+}} & {{f}_{UUU , β + 4}}^{{ur}_{+}} & L & {{f}_{UUU , k}}^{{ur}_{+}}\end{matrix} ]$$

$$\text{?} \text{indicates text missing or illegible when filed}$$

wherein AAA, AAC, UUU were 64 types of trinucleotides formed by 4 types of nucleotides A, C, G, and U of RNA sequences, β represented the distance between the nucleotide at position k and its forward adjacent dinucleotide, 0≤β≤(l−5)/2, β was a finite positive integer, 0≤β≤23 in this example, k represented the position of the trinucleotide, i.e. the position of the first nucleotide of the trinucleotides, β+3≤k≤l−β−2, β+3≤k≤49−β in this example, and k was a finite positive integer,

$${{f}_{AAA , k}}^{{ur}_{+}} , {{f}_{AAC , k}}^{{ur}_{+}} , … , and {{f}_{UUU , k}}^{{ur}_{+}}$$

were the frequencies of trinucleotides AAA, AAC, . . . , or UUU whose nucleotides were at positions k, k+β+1, and k+β+2 of all RNA sequences of positive dataset D⁺, respectively;

The backward trinucleotide position-specific propensity matrix

$$\text{?} {M}_{t}$$

$$\text{?} \text{indicates text missing or illegible when filed}$$

for the positive dataset D⁺ was determined according to the following formula:

$$\text{?} {M}_{t} = [ \begin{matrix}{{f}_{A A A , β + 3}}^{{su}_{+}} & {{f}_{A A A , β + 4}}^{{su}_{+}} & L & {{f}_{AAA , k}}^{{su}_{+}} \\ {{f}_{AAC , β + 3}}^{{su}_{+}} & {{f}_{AAC , β + 4}}^{{su}_{+}} & L & {{f}_{AAC , k}}^{{su}_{+}} \\ M & M & O & M \\ {{f}_{UUU , β + 3}}^{{su}_{+}} & {{f}_{UUU , β + 4}}^{{su}_{+}} & L & {{f}_{UUU , k}}^{{su}_{+}}\end{matrix} ]$$

$$\text{?} \text{indicates text missing or illegible when filed}$$

wherein,

$${{f}_{AAA , k}}^{{su}_{+}} , {{f}_{AAC , k}}^{{su}_{+}} , … , and {{f}_{UUU , k}}^{{su}_{+}}$$

were the occurrence frequencies of trinucleotides AAA, AAC, . . . , and UUU whose nucleotides were at positions k, k−β−1, and k−β−2 of all RNA sequences of positive dataset D⁺, respectively;

The forward trinucleotide position-specific propensity matrix

$$\text{?} {M}_{t}$$

$$\text{?} \text{indicates text missing or illegible when filed}$$

for the negative dataset D⁻ was determined according to the following formula:

$$\text{?} {M}_{t} = [ \begin{matrix}{{f}_{A A A , β + 3}}^{{ur}_{-}} & {{f}_{A A A , β + 4}}^{{ur}_{-}} & L & {{f}_{AAA , k}}^{{ur}_{-}} \\ {{f}_{AAC , β + 3}}^{{ur}_{-}} & {{f}_{AAC , β + 4}}^{{ur}_{-}} & L & {{f}_{AAC , k}}^{{ur}_{-}} \\ M & M & O & M \\ {{f}_{UUU , β + 3}}^{{ur}_{-}} & {{f}_{UUU , β + 4}}^{{ur}_{-}} & L & {{f}_{UUU , k}}^{{ur}_{-}}\end{matrix} ]$$

$$\text{?} \text{indicates text missing or illegible when filed}$$

wherein,

$${{f}_{AAA , k}}^{{ur}_{-}} , {{f}_{AAC , k}}^{{ur}_{-}} , … , and {{f}_{UUU , k}}^{{ur}_{-}}$$

were occurrence frequencies of trinucleotides AAA, AAC, . . . , and UUU whose nucleotides were at positions k, k+β+1, and k+β+2 of all RNA sequences of negative dataset D⁻, respectively;

The backward trinucleotide position-specific propensity matrix

$$\text{?} {M}_{t}$$

$$\text{?} \text{indicates text missing or illegible when filed}$$

for the negative dataset D⁻ was determined according to the following formula:

$$\text{?} {M}_{t} = [ \begin{matrix}{{f}_{A A A , β + 3}}^{{su}_{-}} & {{f}_{A A A , β + 4}}^{{su}_{-}} & L & {{f}_{AAA , k}}^{{su}_{-}} \\ {{f}_{AAC , β + 3}}^{{su}_{-}} & {{f}_{AAC , β + 4}}^{{su}_{-}} & L & {{f}_{AAC , k}}^{{su}_{-}} \\ M & M & O & M \\ {{f}_{UUU , β + 3}}^{{su}_{-}} & {{f}_{UUU , β + 4}}^{{su}_{-}} & L & {{f}_{UUU , k}}^{{su}_{-}}\end{matrix} ]$$

$$\text{?} \text{indicates text missing or illegible when filed}$$

wherein,

$${{f}_{AAA , k}}^{{su}_{-}} , {{f}_{AAC , k}}^{{su}_{-}} , … , and {{f}_{UUU , k}}^{{su}_{-}}$$

were the occurrence frequencies of trinucleotides AAA, AAC, . . . , and UUU whose nucleotides were at positions k, k−β−1, and k−β−2 of all RNA sequences of negative dataset D⁻, respectively;

(4) A value of pointwise joint mutual information of the nucleotides of RNA sequences was determined

(4.1) The value

$${{v}_{k}}^{{r}_{+}}$$

of forward pointwise joint mutual information of the nucleotides of RNA sequences to be encoded in the positive dataset D⁺ was determined according to the following formula:

$${{v}_{k}}^{{r}_{+}} = log \frac{\text{?} {f}_{xyz , k}^{}}{{f}_{x k}^{+} \text{?} {f}_{xyz , k + β + 1}^{}}$$

$$\text{?} \text{indicates text missing or illegible when filed}$$

wherein, x was the nucleotide at position k, x∈{A,C,G,U},

$$\text{?} y$$

$$\text{?} \text{indicates text missing or illegible when filed}$$

was the nucleotide at position k+β+1,

$$\text{?} y ∈ \{ A , C , G , U \} , \text{?} z$$

$$\text{?} \text{indicates text missing or illegible when filed}$$

was the nucleotide at position k+β+2,

$${z}^{\text{?}} ∈ \{ A , C , G , U \} , {{f}_{xyz , k}^{ur}}^{{u}_{+}}$$

$$\text{?} \text{indicates text missing or illegible when filed}$$

was the occurrence frequency of trinucleotide

$${xyz}^{u \text{?}}$$

$$\text{?} \text{indicates text missing or illegible when filed}$$

of all sequences of positive dataset D⁺,

$${{f}_{yz , k + β + 1}^{ur}}^{{u}_{+}}$$

was the occurrence frequency of dinucleotide

$${yz}^{u \text{?}}$$

$$\text{?} \text{indicates text missing or illegible when filed}$$

or all RNA sequences or positive dataset D⁺, and f_{x,k}⁺ was the occurrence frequency of nucleotide of all sequences of positive dataset D⁺.

The value

$${{v}_{k}}^{{s}_{+}}$$

of backward pointwise joint mutual information of nucleotides of RNA sequences to be encoded in the positive dataset D⁺ was determined according to the following formula:

$${{v}_{k}}^{{s}_{+}} = log \frac{{{f}_{xyz , k}^{sus}}^{{su}_{+}}}{{{f}_{x , k}^{+} {f}_{yz , k - β - 1}^{sus}}^{{su}_{+}}}$$

where,

$${y}^{su}$$

was the nucleotide at position k−β−1,

$${y}^{su} ∈ \{ A , C , G , U \} , {z}^{s}$$

was the nucleotide at position k−β−2,

$${z}^{s} ∈ \{ A , C , G , U \} , and {{f}_{x γ z , k}^{sus}}^{{su}_{+}}$$

was the occurrence frequency of trinucleotide

$${x y z}^{sus}$$

of all RNA sequences of positive dataset D⁺,

$${{f}_{yz , k - β - 1}^{sus}}^{{su}_{+}}$$

was the occurrence frequency of dinucleotide

$${yz}^{sus}$$

of all RNA sequences of positive dataset D⁺.

The encoding value vₖ⁺ of pointwise joint mutual information of nucleotide at position k of an RNA sequence to be encoded in the positive dataset D⁺ was defined as the average of the value

$${{v}_{k}}^{{r}_{+}}$$

of forward pointwise joint mutual information and the value

$${{v}_{k}}^{{s}_{+}}$$

of backward pointwise joint mutual information. An RNA sequence with a length of l was encoded into a pointwise mutual information feature vector V⁺ with a length of l−2β−4:

$${V}^{+} = [ {v}_{β + 3}^{+} , {v}_{β + 4}^{+} , L , {v}_{k}^{+} ] \\ \\ \\ {v}_{k}^{+} = \frac{{{v}_{k}}^{{r}_{+}} + {{v}_{k}}^{{s}_{+}}}{2}$$

The value of l was 51 in this example.

(4.2) The value

$$\begin{matrix}{r}_{-} \\ {v}_{k}\end{matrix}$$

of forward pointwise joint mutual information of nucleotides of RNA sequences to be encoded in the negative dataset D⁻ was determined according to the following formula:

$$\begin{matrix}{r}_{-} \\ {v}_{k}\end{matrix} = log \frac{\begin{matrix}{ur}_{-} \\ {f}_{xyz , k}^{ur}\end{matrix}}{{f}_{x , k}^{-} \begin{matrix}{ur}_{-} \\ {f}_{yz , k + β + 1}^{ur}\end{matrix}}$$

wherein, x was the nucleotide at position k, xE{A,C,G,U},

$$\begin{matrix}u \\ y\end{matrix}$$

was the nucleotide at position k+β+1,

$$\begin{matrix}u \\ y ∈ \{ A , C , G , U \} ,\end{matrix} \begin{matrix}r \\ z\end{matrix}$$

was the nucleotide at position k+β+2,

$$\begin{matrix}r \\ z ∈ \{ A , C , G , U \}\end{matrix} , and \begin{matrix}{ur}_{-} \\ {f}_{xyz , k}^{ur}\end{matrix}$$

was the occurrence frequency of trinucleotide

$$\begin{matrix}ur \\ xyz\end{matrix}$$

of all sequences of negative dataset D⁻,

$$\begin{matrix}{ur}_{-} \\ {f}_{yz , k + β + 1}^{ur}\end{matrix}$$

was the occurrence frequency of dinucleotide

$$\begin{matrix}ur \\ yz\end{matrix}$$

of all sequences of negative dataset D⁻, and f_{x,k}⁻ was the occurrence frequency of nucleotide x of all sequences of negative dataset D⁻.

The value

$$\begin{matrix}{s}_{-} \\ {v}_{k}\end{matrix}$$

of backward pointwise joint mutual information of nucleotides of RNA sequences to be encoded in negative dataset D⁻ was determined according to the following formula:

$$\begin{matrix}{s}_{-} \\ {v}_{k}\end{matrix} = log \frac{\begin{matrix}{su}_{-} \\ {f}_{xyz , k}^{sus}\end{matrix}}{\begin{matrix}{su}_{-} \\ {f}_{x , k}^{-} {f}_{yz , k - β - 1}^{sus}\end{matrix}}$$

wherein, nucleotide x was at position k, and nucleotide

$$\begin{matrix}su \\ y\end{matrix}$$

was at position k−β−1, and nucleotide

$$\begin{matrix}s \\ z\end{matrix}$$

was at position k−β−2,

$$\begin{matrix}{su}_{-} \\ {f}_{xyz , k}^{sus}\end{matrix}$$

was the occurrence frequency of trinucleotide

$$\begin{matrix}sus \\ xyz\end{matrix}$$

of all RNA sequences of negative dataset D⁻,

$$\begin{matrix}{su}_{-} \\ {f}_{yz , k - β - 1}^{sus}\end{matrix}$$

was the occurrence frequency of dinucleotide

$$\begin{matrix}sus \\ yz\end{matrix}$$

of all sequences of negative dataset D⁻.

The encoding value vₖ⁻ of pointwise joint mutual information of the nucleotide at position k of an RNA sequence to be encoded in the negative dataset D⁻ was defined as the average of the value

$${{v}_{k}}^{{r}_{-}}$$

of forward pointwise joint mutual information and the value

$${{v}_{k}}^{{s}_{-}}$$

of backward pointwise joint mutual information, and an RNA sequence with a length of l was encoded into a pointwise mutual information feature vector V⁻ with a length of l−2β−4:

$$V = [ {v}_{β + 3}^{-} , {v}_{β + 4}^{-} , L , {v}_{k}^{-} ] \\ {v}_{k} = \frac{{{v}_{k}}^{{r}_{-}} + {{v}_{k}}^{{s}_{-}}}{2}$$

The value of l was 51 in this example.

(4.3) The feature vector V of an RNA sequence to be encoded with a given length l was determined by corresponding element of vector V⁺ minus that of V⁻:

V=[V_{β+3}, V_{β+4}, . . . , Vₖ]

Vₖ=vₖ⁺−vₖ⁻;

(5) Concatenating features

when the value of parameter β was 0, the feature vector V(0) was [V₃, V₄, V₅, . . . , Vₗ₋₃, Vₗ₋₂], and the number of elements was l−4; when the value of β was 1, the feature vector V(1) was [V₄, V₅, V₆, . . . , Vₗ₋₄, Vₗ₋₃], and the number of elements was l−6, . . . , and when the value of β was (l−7)/2, the feature vector V((l−7)/2) was [V_{(l-1)/2}, V_{(l-1)/2}, V_{(l+3)/2}], the number of elements was 3; when the value of β was (l−5)/2, the feature vector V((l−5)/2) was [V_{(l+1)/2}], and the number of elements was 1; the feature vectors determined by different values of the parameter β were concatenated into a high-dimensional feature vector [V(0), V(1), . . . , V((l−7)/2), V((l−5)/2)] with (l−3)²/4 elements, the value of l was 51 in this example.

(6) Encoding the RNA sequences

The RNA sequence dataset D was encoded into a numerical dataset D′ by adopting the above step (1)-step (5),

$${D}^{′} ∈ {R}^{s \times \frac{{( l - 3 )}^{2}}{4}} ,$$

where s was a number of samples of the numerical dataset D′, and s was a finite positive integer, the value of s was 2614 in this example, and (l−3)²/4 was a feature number of the numerical data set D′. The encoding of RNA sequences was completed.

The RNA sequence encoding method of Example 2 was compared with PSNP (position-specific nucleotide propensities), PSDP (position-specific dinucleotide propensities), KNF (K-nucleotide frequencies), KSNPF (K spaced nucleotide pair frequencies), NPPS (nucleotide pair position specificity), PBE (positional binary encoding) and NCPNC (nucleotide chemical property and nucleotide composition) encoding methods which were for identifying the RNA N⁶-methyladenosine sites in Saccharomyces cerevisiae RNA sequences by the performance of support vector machine models constructed using each encoding method. The average classification accuracy, sensitivity, specificity, MCC (Mathew's Correlation coefficient), AUROC (Area under the receiver operating characteristic curve) and AUPRC (Area under the precision recall curve) of 10-fold cross-validation method were used to evaluate each method. The experimental method was as follows:

1. The RNA sequences of N⁶-methyladenosine of Saccharomyces cerevisiae were encoded according to the method of Example 2;

2. Normalizing the dataset

The numerical dataset D′ was normalized by the maximum-minimum method according to the following formula:

$${g}_{m , n}^{′} = \frac{{g}_{m , n} - min ( {g}_{n} )}{max ( {g}_{n} ) - min ( {g}_{n} )}$$

wherein g_{m,n} was the n-th feature value of the m-th sample of the numerical dataset D′, the normalized value of g_{m,n} was g′_{m,n}, max(gₙ) and min(gₙ) represent the maximum and minimum feature values of the n-th column of the numerical dataset D′, 1≤m≤s, 1≤n≤(l−1)²/4, m and n were finite positive integers, the value of l in this example was 51, and the value of s was 2614.

3. Partitioning dataset

The normalized numerical dataset D′ was partitioned into 10 folds by using the K-fold cross-validation method (K=10), one fold was taken as the test dataset D′_{Te}, and the remaining nine folds are taken as the training dataset D′_{Tr}, till each fold was taken as the test dataset, so there were 10 runs in total. The ratio of the training dataset D′_{Tr} to the test dataset D′_{Te} in each run was 9:1.

4. Training and testing the model

The support vector machine model was trained using training dataset D′_{Tr}, and the performance of the support vector machine model is tested by the test dataset D′_{Te}.

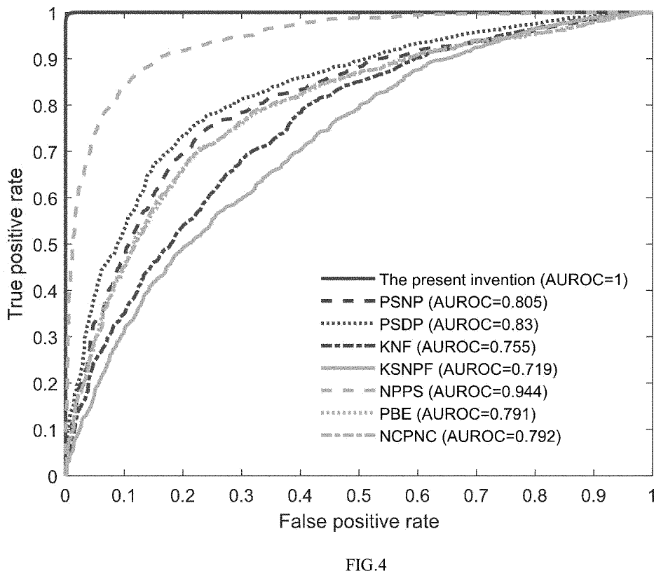

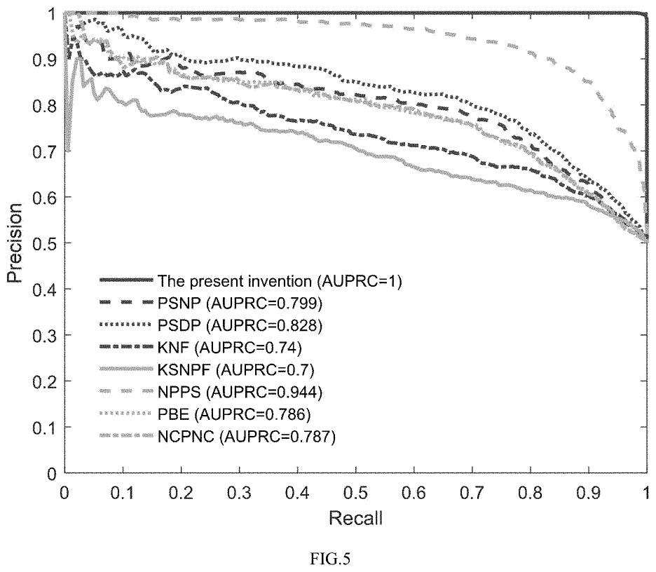

The RNA N⁶-methyladenosine sites in the Saccharomyces cerevisiae RNA sequences were identified by performing the same operation on the seven compared RNA sequence encoding methods according to steps 2-4 of the experimental methods. The experimental results of classification accuracy, sensitivity, specificity and MCC were shown in Table 2, the experimental results of AUROC were shown in FIG. 4, and the experimental results of AUPRC were shown in FIG. 5.

TABLE 2
Comparison of experimental results between the
method of Example 2 and other seven methods

| | Evaluation criterion | | | |
|---|---|---|---|---|
| Encoding method | Accuracy | Sensitivity | Specificity | MCC |
| The present invention | 0.995 | 0.996 | 0.994 | 0.990 |
| PSNP | 0.747 | 0.751 | 0.743 | 0.495 |
| PSDP | 0.766 | 0.764 | 0.769 | 0.534 |
| KNF | 0.692 | 0.741 | 0.643 | 0.387 |
| KSNPF | 0.651 | 0.712 | 0.591 | 0.307 |
| NPPS | 0.874 | 0.884 | 0.864 | 0.749 |
| PBE | 0.727 | 0.727 | 0.728 | 0.456 |
| NCPNC | 0.731 | 0.735 | 0.726 | 0.463 |

As shown in Table 2, the accuracy, sensitivity, specificity and MCC for identifying the RNA N⁶-methyladenosine sites in Saccharomyces cerevisiae RNA sequences through the support vector machine model constructed based on the RNA sequence encoding method of the present disclosure were 0.995, 0.996, 0.994 and 0.990, respectively, which were much higher than those of the other seven compared encoding methods.

As shown in FIG. 4, the value of AUROC for identifying the RNA N⁶-methyladenosine sites in Saccharomyces cerevisiae RNA sequences through the support vector machine model constructed based on the RNA sequence encoding method of the present disclosure was the maximum value of 1, which was much higher than that of the other seven compared encoding methods.

As shown in FIG. 5, the value of AUPRC for identifying the RNA N⁶-methyladenosine sites in Saccharomyces cerevisiae RNA sequences through the support vector machine model constructed based on the RNA sequence encoding method of the present disclosure was the maximum value of 1, which was much higher than that of the other seven compared encoding methods.

1. A method for encoding DNA/RNA sequences based on bidirectional trinucleotide position-specific propensities and pointwise joint mutual information, comprising the following steps:
(1) constructing a nucleotide position-specific propensity matrix of DNA/RNA sequences:
giving a dataset D of DNA/RNA sequences, the dataset consists of a positive dataset D⁺ and a negative dataset D⁻;
determining a nucleotide position-specific propensity matrix M_{S}⁺ for the positive dataset according to the following formula:
$${M}_{s}^{+} = [ \begin{matrix}{f}_{A , 1}^{+} & {f}_{A , 2}^{+} & L & {f}_{A , i}^{+} \\ {f}_{C , 1}^{+} & {f}_{C , 2}^{+} & L & {f}_{C , i}^{+} \\ {f}_{G , 1}^{+} & {f}_{G , 2}^{+} & L & {f}_{G , i}^{+} \\ {f}_{X , 1}^{+} & {f}_{X , 2}^{+} & L & {f}_{X , i}^{+}\end{matrix} ]$$
wherein, A, C, G and X are 4 types of nucleotides of DNA/RNA, wherein, X represents nucleotide T in DNA, and represents nucleotide U in RNA, and i represents a position of nucleotide, 1≤i≤l, and i is a finite positive integer, l represents a length of a DNA/RNA sequence, and l is an odd number, f_{A,i}⁺, f_{C,i}⁺, f_{G,i}⁺ and f_{X,i}⁺ are occurrence frequencies of nucleotides A, C, G and X at position i of all sequences of positive dataset D⁺, respectively;
determining a nucleotide position-specific propensity matrix M_{S}⁻ of the negative dataset D⁻ according to the following formula:
$${M}_{s}^{-} = [ \begin{matrix}{f}_{A , 1}^{-} & {f}_{A , 2}^{-} & L & {f}_{A , i}^{-} \\ {f}_{C , 1}^{-} & {f}_{C , 2}^{-} & L & {f}_{C , i}^{-} \\ {f}_{G , 1}^{-} & {f}_{G , 2}^{-} & L & {f}_{G , i}^{-} \\ {f}_{X , 1}^{-} & {f}_{X , 2}^{-} & L & {f}_{X , i}^{-}\end{matrix} ]$$
wherein f_{A,i}⁻, f_{C,i}⁻, f_{G,i}⁻ and f_{X,i}⁻ are occurrence frequencies of nucleotides A, C, G and X at position i of all sequences of negative dataset D⁻, respectively;
(2) constructing a bidirectional dinucleotide position-specific propensity matrix of DNA/RNA sequences:
determining a forward dinucleotide position-specific propensity matrix
$$\text{?} {M}_{d}$$
$$\text{?} \text{indicates text missing or illegible when filed}$$
for the positive dataset D⁺ according to the following formula:
$$\text{?} {M}_{d} = [ \begin{matrix}\text{?} {f}_{AA , 1} & \text{?} {f}_{AA , 2} & L & \text{?} {f}_{AA , j} \\ \text{?} {f}_{A C , 1} & \text{?} {f}_{A C , 2} & L & \text{?} {f}_{A C , j} \\ M & M & O & M \\ \text{?} {f}_{xx , 1} & \text{?} {f}_{xx , 2} & L & \text{?} {f}_{xx , j}\end{matrix} ]$$
$$\text{?} \text{indicates text missing or illegible when filed}$$
wherein, AA, AC, . . . , and XX are 16 types of dinucleotides formed by the 4 types of nucleotides A, C, G, and X of DNA/RNA, j represents position of a dinucleotide, 2≤j≤l−1, and j is a finite positive integer,
$$\text{?} {f}_{AA , j} , \text{?} {f}_{A C , j} , … , and \text{?} {f}_{xx , j}$$
$$\text{?} \text{indicates text missing or illegible when filed}$$
are occurrence frequencies of dinucleotides AA, AC, . . . , and XX of all sequences of positive dataset D⁺, respectively;
determining a backward dinucleotide position-specific propensity matrix
$$\text{?} {M}_{d}$$
$$\text{?} \text{indicates text missing or illegible when filed}$$
for the positive dataset D⁺ according to the following formula:
$$\text{?} {M}_{d} = [ \begin{matrix}\text{?} {f}_{AA , 2} & \text{?} {f}_{AA , 3} & L & \text{?} {f}_{AA , j} \\ \text{?} {f}_{A C , 2} & \text{?} {f}_{A C , 3} & L & \text{?} {f}_{A C , j} \\ M & M & O & M \\ \text{?} {f}_{xx , 2} & \text{?} {f}_{xx , 3} & L & \text{?} {f}_{xx , j}\end{matrix} ]$$
$$\text{?} \text{indicates text missing or illegible when filed}$$
wherein,
$$\text{?} {f}_{AA , j} , \text{?} {f}_{A C , j} , … , and \text{?} {f}_{xx , j}$$
$$\text{?} \text{indicates text missing or illegible when filed}$$
are occurrence frequencies of dinucleotides AA, AC, . . . , and XX of all sequences of positive dataset D⁺, respectively, wherein the two nucleotides of these dinucleotides are at positions j and j−1, respectively;
determining a forward dinucleotide position-specific propensity matrix
$$\begin{matrix}\text{?} \\ {M}_{d}\end{matrix}$$
$$\text{?} \text{indicates text missing or illegible when filed}$$
for the negative dataset D⁻ according to the following formula:
$$\text{?} {M}_{d} = [ \begin{matrix}\text{?} {f}_{AA , 2} & \text{?} {f}_{AA , 2} & L & \text{?} {f}_{AA , j} \\ \text{?} {f}_{AA , 2} & \text{?} {f}_{A C , 3} & L & \text{?} {f}_{A C , j} \\ M & M & O & M \\ \text{?} {f}_{xx , 2} & \text{?} {f}_{xx , 3} & L & \text{?} {f}_{xx , j}\end{matrix} ]$$
$$\text{?} \text{indicates text missing or illegible when filed}$$
wherein
$$\text{?} {f}_{AA , j} , \text{?} {f}_{A C , j} , … , and \text{?} {f}_{xx , j}$$
$$\text{?} \text{indicates text missing or illegible when filed}$$
wherein are occurrence frequencies of dinucleotides AA, AC, . . . , and XX of all sequences of negative dataset D⁻, respectively, and the two nucleotides of these dinucleotides are at positions j and j+1, respectively;
determining a backward dinucleotide position-specific propensity matrix
$$\begin{matrix}\text{?} \\ {M}_{d}\end{matrix}$$
$$\text{?} \text{indicates text missing or illegible when filed}$$
for the negative dataset D⁻ according to the following formula:
$$\begin{matrix}\text{?} \\ {M}_{d}\end{matrix} = [ \begin{matrix}\begin{matrix}\text{?} \\ {f}_{AA , 2}\end{matrix} & \begin{matrix}\text{?} \\ {f}_{AA , 3}\end{matrix} & L & \begin{matrix}\text{?} \\ {f}_{AA , j}\end{matrix} \\ \begin{matrix}\text{?} \\ {f}_{AC , 2}\end{matrix} & \begin{matrix}\text{?} \\ {f}_{AC , 3}\end{matrix} & L & \begin{matrix}\text{?} \\ {f}_{AC , j}\end{matrix} \\ M & M & O & M \\ \begin{matrix}\text{?} \\ {f}_{XX , 2}\end{matrix} & \begin{matrix}\text{?} \\ {f}_{XX , 3}\end{matrix} & L & \begin{matrix}\text{?} \\ {f}_{XX , j}\end{matrix}\end{matrix} ]$$
$$\text{?} \text{indicates text missing or illegible when filed}$$
wherein,
$$\begin{matrix}\text{?} \\ {f}_{AA , j}\end{matrix} , \begin{matrix}\text{?} \\ {f}_{AC , j}\end{matrix} , … , and \begin{matrix}\text{?} \\ {f}_{XX , j}\end{matrix}$$
$$\text{?} \text{indicates text missing or illegible when filed}$$
are occurrence frequencies of dinucleotides AA, AC, . . . , and XX of all sequences of negative dataset, respectively, and their two nucleotides are at positions j and j−1, respectively;
(3) constructing a bidirectional trinucleotide position-specific propensity matrix of DNA/RNA sequences:
determining a forward trinucleotide position-specific propensity matrix
$$\begin{matrix}{\text{?}}_{+} \\ {M}_{t}\end{matrix}$$
$$\text{?} \text{indicates text missing or illegible when filed}$$
for the positive dataset D⁺ according to the following formula:
$$\begin{matrix}\text{?} \\ {M}_{t}\end{matrix} = [ \begin{matrix}\begin{matrix}\text{?} \\ {f}_{AAA , β + 3}\end{matrix} & \begin{matrix}\text{?} \\ {f}_{AAA , β + 4}\end{matrix} & L & \begin{matrix}\text{?} \\ {f}_{AAA , k}\end{matrix} \\ \begin{matrix}\text{?} \\ {f}_{AAC , β + 3}\end{matrix} & \begin{matrix}\text{?} \\ {f}_{AAC , β + 4}\end{matrix} & L & \begin{matrix}\text{?} \\ {f}_{AAC , k}\end{matrix} \\ M & M & O & M \\ \begin{matrix}\text{?} \\ {f}_{XXX , β + 3}\end{matrix} & \begin{matrix}\text{?} \\ {f}_{XXX , β + 4}\end{matrix} & L & \begin{matrix}\text{?} \\ {f}_{XXX , k}\end{matrix}\end{matrix} ]$$
$$\text{?} \text{indicates text missing or illegible when filed}$$
wherein AAA, AAC, . . . , XXX are 64 types of trinucleotides formed by 4 types of nucleotides A, C, G, and X of DNA/RNA, β represents a distance between the nucleotide at position k and its forward adjacent dinucleotide, 0≤β≤(l−5)/2, and β is a finite positive integer, k represents a position of trinucleotide, β+3≤k≤l−β−2, and k is a finite positive integer,
$$\begin{matrix}\text{?} \\ {f}_{AAA , k}\end{matrix} , \begin{matrix}\text{?} \\ {f}_{AAC , k}\end{matrix} , … , and \begin{matrix}\text{?} \\ {f}_{XXX , k}\end{matrix}$$
$$\text{?} \text{indicates text missing or illegible when filed}$$
are occurrence frequencies of trinucleotides AAA, AAC, . . . , and XXX of all sequences of positive dataset D⁺, respectively;
determining a backward trinucleotide position-specific propensity matrix
$$\begin{matrix}\text{?} \\ {M}_{t}\end{matrix}$$
$$\text{?} \text{indicates text missing or illegible when filed}$$
for the positive dataset D⁺ according to the following formula:
$$\begin{matrix}\text{?} \\ {M}_{t}\end{matrix} = [ \begin{matrix}\begin{matrix}\text{?} \\ {f}_{AAA , β + 3}\end{matrix} & \begin{matrix}\text{?} \\ {f}_{AAA , β + 4}\end{matrix} & L & \begin{matrix}\text{?} \\ {f}_{AAA , k}\end{matrix} \\ \begin{matrix}\text{?} \\ {f}_{AAC , β + 3}\end{matrix} & \begin{matrix}\text{?} \\ {f}_{AAC , β + 4}\end{matrix} & L & \begin{matrix}\text{?} \\ {f}_{AAC , k}\end{matrix} \\ M & M & O & M \\ \begin{matrix}\text{?} \\ {f}_{XXX , β + 3}\end{matrix} & \begin{matrix}\text{?} \\ {f}_{XXX , β + 4}\end{matrix} & L & \begin{matrix}\text{?} \\ {f}_{XXX , k}\end{matrix}\end{matrix} ]$$
$$\text{?} \text{indicates text missing or illegible when filed}$$
wherein,
$$\begin{matrix}\text{?} \\ {f}_{AAA , k}\end{matrix} , \begin{matrix}\text{?} \\ {f}_{AAC , k}\end{matrix} , … , and \begin{matrix}\text{?} \\ {f}_{XXX , k}\end{matrix}$$
$$\text{?} \text{indicates text missing or illegible when filed}$$
are occurrence frequencies of trinucleotides AAA, AAC, . . . , and XXX of all sequences of positive dataset D⁺, respectively, and a first, second and third nucleotide of these trinucleotides are at positions k, k−β−1, and k−β−2, respectively;
determining a forward trinucleotide position-specific propensity matrix
$$\begin{matrix}\text{?} \\ {M}_{t}\end{matrix}$$
$$\text{?} \text{indicates text missing or illegible when filed}$$
for the negative dataset D⁻ according to the following formula:
$$\begin{matrix}\text{?} \\ {M}_{t}\end{matrix} = [ \begin{matrix}\begin{matrix}\text{?} \\ {f}_{AAA , β + 3}\end{matrix} & \begin{matrix}\text{?} \\ {f}_{AAA , β + 4}\end{matrix} & L & \begin{matrix}\text{?} \\ {f}_{AAA , k}\end{matrix} \\ \begin{matrix}\text{?} \\ {f}_{AAC , β + 3}\end{matrix} & \begin{matrix}\text{?} \\ {f}_{AAC , β + 4}\end{matrix} & L & \begin{matrix}\text{?} \\ {f}_{AAC , k}\end{matrix} \\ M & M & O & M \\ \begin{matrix}\text{?} \\ {f}_{XXX , β + 3}\end{matrix} & \begin{matrix}\text{?} \\ {f}_{XXX , β + 4}\end{matrix} & L & \begin{matrix}\text{?} \\ {f}_{XXX , k}\end{matrix}\end{matrix} ]$$
$$\text{?} \text{indicates text missing or illegible when filed}$$
wherein,
$$\begin{matrix}\text{?} \\ {f}_{AAA , k}\end{matrix} , \begin{matrix}\text{?} \\ {f}_{AAC , k}\end{matrix} , … , and \begin{matrix}\text{?} \\ {f}_{XXX , k}\end{matrix}$$
$$\text{?} \text{indicates text missing or illegible when filed}$$
are occurrence frequencies of trinucleotides AAA, AAC, . . . , and XXX of all sequences of negative dataset D⁻, respectively, and a first, second and third nucleotide of these trinucleotides are at positions k, k+β+1, and k+β+2, respectively;
determining a backward trinucleotide position-specific propensity matrix
$$s \text{?} {M}_{t}$$
$$\text{?} \text{indicates text missing or illegible when filed}$$
for the negative dataset D⁻ according to the following formula:
$$\begin{matrix}\text{?} \\ {M}_{t}\end{matrix} = [ \begin{matrix}\begin{matrix}\text{?} \\ {f}_{AAA , β + 3}\end{matrix} & \begin{matrix}\text{?} \\ {f}_{AAA , β + 4}\end{matrix} & L & \begin{matrix}\text{?} \\ {f}_{AAA , k}\end{matrix} \\ \begin{matrix}\text{?} \\ {f}_{AAC , β + 3}\end{matrix} & \begin{matrix}\text{?} \\ {f}_{AAC , β + 4}\end{matrix} & L & \begin{matrix}\text{?} \\ {f}_{AAC , k}\end{matrix} \\ M & M & O & M \\ \begin{matrix}\text{?} \\ {f}_{XXX , β + 3}\end{matrix} & \begin{matrix}\text{?} \\ {f}_{XXX , β + 4}\end{matrix} & L & \begin{matrix}\text{?} \\ {f}_{XXX , k}\end{matrix}\end{matrix} ]$$
$$\text{?} \text{indicates text missing or illegible when filed}$$
wherein,
$$\begin{matrix}\text{?} \\ {f}_{AAA , k}\end{matrix} , \begin{matrix}\text{?} \\ {f}_{AAC , k}\end{matrix} , … , and \begin{matrix}\text{?} \\ {f}_{XXX , k}\end{matrix}$$
$$\text{?} \text{indicates text missing or illegible when filed}$$
are occurrence frequencies of trinucleotides AAA, AAC, . . . , and XXX of all sequences of negative dataset D⁻, respectively, and a first and second and third nucleotide of these trinucleotides are at positions k, k−β−1, and k−β−2, respectively;
(4) determining a value of pointwise joint mutual information of the nucleotides of DNA/RNA sequences:
(4.1) determining a value
$$\begin{matrix}{r}_{+} \\ {v}_{k}\end{matrix}$$
of forward pointwise joint mutual information of nucleotides of DNA/RNA sequences to be encoded in the positive dataset D⁺ according to the following formula:
$$\begin{matrix}{r}_{+} \\ {v}_{k}\end{matrix} = log \frac{\begin{matrix}{ur}_{+} \\ {f}_{xyz , k}^{ur}\end{matrix}}{\begin{matrix}{ur}_{+} \\ {f}_{x , k}^{+} {f}_{yz , k + β + 1}^{ur}\end{matrix}}$$
wherein, x is a nucleotide at position k, x∈{A, C, G, X},
$$\begin{matrix}u \\ y\end{matrix}$$
is a nucleotide at position k+β+1,
$$\text{?} y ∈ \{ A < C < G < X \} , z \text{?}$$
$$\text{?} \text{indicates text missing or illegible when filed}$$
is a nucleotide at position k+β+2,
$${z}^{1} ∈ \{ A , C , G , X \} and \begin{matrix}{ur}_{+} \\ {f}_{xyz , k}^{ur}\end{matrix}$$
is an occurrence frequency of trinucleotide
$$\begin{matrix}u 1 \\ xyz\end{matrix}$$
of all sequences of positive dataset D⁺,
$$\begin{matrix}{ur}_{+} \\ {f}_{yz , k + β + 1}^{ur}\end{matrix}$$
is an occurrence frequency of dinucleotide
$$\begin{matrix}u 1 \\ yz\end{matrix}$$
of all sequences of positive dataset D⁺, and f_{x,k}⁺ is an occurrence frequency of nucleotide x of all sequences of positive dataset D⁺;
determining a value
$${{v}_{k}}^{{s}_{+}}$$
of backward pointwise joint mutual information of nucleotides of DNA/RNA sequences to be encoded in the positive dataset D⁺ according to the following formula:
$${{v}_{k}}^{{s}_{+}} = log \frac{{{f}_{x , y , z , k}}^{{su}_{+}} \text{?}}{{{f}_{x , k}^{+}}^{{su}_{+}} {f}_{yz , k - β - 1} \text{?}}$$
$$\text{?} \text{indicates text missing or illegible when filed}$$
wherein, x is a nucleotide at position k, x∈{A, C, G, X},
$$\text{?} y$$
$$\text{?} \text{indicates text missing or illegible when filed}$$
is a nucleotide at position k−β−1,
$$\text{?} y ∈ \{ A , C , G , X \} z \text{?}$$
$$\text{?} \text{indicates text missing or illegible when filed}$$
is a nucleotide at position k−β−2,
$$\begin{matrix}s & {su}_{+} \\ z ∈ \{ A , C , G , X \} b , & {f}_{xyz , k}^{s}\end{matrix}$$
is an occurrence frequency of trinucleotide
$$\text{?} xyz$$
$$\text{?} \text{indicates text missing or illegible when filed}$$
of all sequences of positive dataset D⁺,
$$\text{?} {f}_{y z , k - β - 1}$$
$$\text{?} \text{indicates text missing or illegible when filed}$$
is an occurrence frequency of dinucleotide
$$\begin{matrix}\text{?} \\ yz\end{matrix}$$
$$\text{?} \text{indicates text missing or illegible when filed}$$
of all sequences of positive dataset D⁺;
the encoding value vₖ⁺ of pointwise joint mutual information of the nucleotide at position k of DNA/RNA sequences to be encoded in the positive dataset D⁺ is defined as the average of the value
$${{v}_{k}}^{{r}_{+}}$$
of forward pointwise joint mutual information and the value
$${{v}_{k}}^{{s}_{+}}$$
of backward pointwise joint mutual information, and a DNA/RNA sequence with a length of l is encoded into a pointwise mutual information feature vector V⁺ with a length of l−2,β−4:
$${V}^{+} = [ {v}_{β + 3}^{+} , {v}_{β + 4}^{+} , L , {v}_{k}^{+} ] \\ {v}_{k}^{+} = \frac{{{v}_{k}}^{{r}_{+}} + {{v}_{k}}^{{s}_{+}}}{2}$$
(4.2) determining a value
$${{v}_{k}}^{{r}_{-}}$$
of forward pointwise joint mutual information of nucleotides of DNA/RNA sequences to be encoded in the negative dataset D⁻ according to the following formula:
$${{v}_{k}}^{{r}_{-}} = log \frac{\text{?} {f}_{xyz , k}}{\text{?} {f}_{x , k}^{-} \text{?} {f}_{yz , k + β + 1}}$$
$$\text{?} \text{indicates text missing or illegible when filed}$$
wherein, x,
$$\text{?} y and \text{?} z$$
$$\text{?} \text{indicates text missing or illegible when filed}$$
are nucleotides at positions k, k+β+1 and k+β+2 of all sequences of negative dataset D⁻, respectively,
$$\text{?} x , y , z ∈ \{ A , C , G , X \} , and \text{?} {f}_{xyz , k}$$
$$\text{?} \text{indicates text missing or illegible when filed}$$
is an occurrence frequency of trinucleotide
$$\text{?} xyz$$
$$\text{?} \text{indicates text missing or illegible when filed}$$
of all sequences of negative dataset D⁻,
$$\text{?} {f}_{yz , k + β + 1}$$
$$\text{?} \text{indicates text missing or illegible when filed}$$
is an occurrence frequency of dinucleotide
$$\text{?} yz$$
$$\text{?} \text{indicates text missing or illegible when filed}$$
of all sequences of negative dataset D⁻, and f_{x,k}⁻ is an occurrence frequency of nucleotide x of all sequences of negative dataset D⁻;
determining a value
$${{v}_{k}}^{{s}_{-}}$$
of backward pointwise joint mutual information of nucleotides of DNA/RNA sequences to be encoded in the negative dataset D⁻ according to the following formula:
$${{v}_{k}}^{{s}_{-}} = log \frac{\text{?} {f}_{x y z , k}}{\text{?} {f}_{x , k}^{-} \text{?} {f}_{y z , k - β - 1}}$$
$$\text{?} \text{indicates text missing or illegible when filed}$$
wherein,
$$\text{?} x , y , z$$
$$\text{?} \text{indicates text missing or illegible when filed}$$
are nucleotides at positions k, k−β−1 and k−β−2 of all sequence samples of negative dataset D⁻, respectively,
$${x , y , z ∈}^{\text{?}} \{ A , C , G , X \} , and {{f}^{\text{?}}}_{{xyz , k}^{\text{?}}}$$
$$\text{?} \text{indicates text missing or illegible when filed}$$
is an occurrence frequency of trinucleotide
$${xyz}^{\text{?}}$$
$$\text{?} \text{indicates text missing or illegible when filed}$$
of all sequence samples of negative dataset D⁻,
$${{f}^{\text{?}}}_{{yz}^{\text{?}} , k - β - 1}$$
$$\text{?} \text{indicates text missing or illegible when filed}$$
is an occurrence frequency of dinucleotide
$${yz}^{\text{?}}$$
$$\text{?} \text{indicates text missing or illegible when filed}$$
of all sequences of negative dataset D⁻;
the encoding value vₖ⁻ of pointwise joint mutual information of the nucleotide at position k of DNA/RNA sequences to be encoded in the negative dataset D⁻ is defined as an average of the value
$$\begin{matrix}r \_ \\ {v}_{k}\end{matrix}$$
of forward pointwise joint mutual information and the value
$$\begin{matrix}{s}_{-} \\ {v}_{k}\end{matrix}$$
of backward pointwise joint mutual information, and a DNA/RNA sequence with a length of l is encoded into a pointwise mutual information feature vector V⁻ with a length of l−2β−4:
$${V}^{-} = [ {v}_{β + 3}^{-} , {v}_{β + 4}^{-} , L , {v}_{k}^{-} ] \\ {v}_{k}^{-} = \frac{\begin{matrix}{r}_{-} \\ {v}_{k}\end{matrix} + \begin{matrix}{s}_{-} \\ {v}_{k}\end{matrix}}{2}$$
(4.3) determining a feature vector V of a DNA/RNA sequence to be encoded with a given length l by corresponding element of vector V⁺ minus that of V⁻:
V=[V_{β+3}, V_{β+4}, . . . , Vₖ]
Vₖ=vₖ⁺−vₖ⁻
(5) concatenating features
when value of parameter β is 0, the feature vector V(0) is [V₃, V₄, V₅, . . . , Vₗ₋₃, Vₗ₋₂], and the number of elements is l−4; when value of β is 1, the feature vector V(1) is [V₄, V₅, V₆, . . . , Vₗ₋₄, Vₗ₋₃], and the number of elements is l−6, . . . , and when value of β is (l−7)/2, the feature vector V((l−7)/2) is [V_{(l-1)/2}, V_{(l+1)/2}, V_{(l+3)/2}], the number of elements is 3; when value of β is (l−5)/2, the feature vector V((l−5)/2) is [V_{(l+1)/2}], and the number of elements is 1; concatenating the feature vectors determined by different values of parameter β into a high-dimensional feature vector [V(0), V(1), . . . , V((l−7)/2), V((l−5)/2)] with (l−3)²/4 elements;
(6) encoding DNA/RNA sequences
encoding the DNA/RNA sequence dataset D into a numerical dataset D′ by performing the above step (1)-step (5),
$${D}^{′} ∈ {R}^{s \times \frac{{( l - 3 )}^{2}}{4}} ,$$
where s is a number of samples in the numerical dataset D′, and s is a finite positive integer, and (l−3)²/4 is a feature number of the numerical data set D′.
